SEQUENCE LISTING (22 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, Artificial Sequence [sequence omitted]

SEQ ID NO: 5 - protein, 107 aa, Artificial Sequence [sequence omitted]

SEQ ID NO: 6 - protein, 5 aa, Artificial Sequence
  daratumumab HCDR1
SFAMS

SEQ ID NO: 7 - protein, 17 aa, Artificial Sequence
  daratumumab HCDR2
AISGSGGGTYYADSVKG

SEQ ID NO: 8 - protein, 13 aa, Artificial Sequence
  daratumumab HCDR3
DKILWFGEPVFDY

SEQ ID NO: 9 - protein, 11 aa, Artificial Sequence
  daratumumab LCDR1
RASQSVSSYLA

SEQ ID NO: 10 - protein, 7 aa, Artificial Sequence
  daratumumab LCDR2
DASNRAT

SEQ ID NO: 11 - protein, 10 aa, Artificial Sequence
  daratumumab LCDR3
QQRSNWPPTF

SEQ ID NO: 12 - protein, 452 aa, Artificial Sequence
  daratumumab HC
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial Sequence
  daratumumab LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, Artificial Sequence
  mAb 003 VH
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, Artificial Sequence
  mAb 003 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, Artificial Sequence
  mAb 024 VH
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, Artificial Sequence
  mAb 024 VL
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, Artificial Sequence
  MOR-202 VH
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, Artificial Sequence
  MOR-202 VL
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, Artificial Sequence [sequence omitted]

SEQ ID NO: 21 - protein, 107 aa, Artificial Sequence [sequence omitted]

SEQ ID NO: 22 - protein, 509 aa, Artificial Sequence
  recombinant huPH20
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

(19) United States
(12) Patent Application Publication
(10) Pub. No.: US 2018/0117150 A1
(43) Pub. Date: May 3, 2018

(54) Combination Therapies for CD38-Positive Hematological Malignances with ANTI-CD38 Antibodies and Cyclophosphamide

(71) Applicant: Janssen Biotech, Inc., Horsham, PA (US)

(72) Inventors: Michael O'Dwyer, Galway (IE); Aideen Ryan, Galway (IE)

(21) Appl. No.: 15/798,670

(22) Filed: Oct. 31, 2017

Related U.S. Application Data
(60) Provisional application No. 62/415,749, filed on Nov. 1, 2016.

(51) Int. Cl. A61K 39/395; C07K 16/28; A61K 31/675; A61K 38/05; A61K 31/454; A61K 31/573; A61K 38/47; A61K 9/00

(52) U.S. Cl. CPC A61K 39/39558; C07K 16/2896; A61K 31/675; A61K 38/05; A61K 2039/545; A61K 31/573; A61K 38/47; A61K 9/0019; C12Y 302/01035; A61K 31/454

(57) ABSTRACT

Provided are combination therapies comprising an anti-CD38 antibody and cyclophosphamide for CD38-positive hematological malignancies.

### FIELD OF THE INVENTION

Disclosed are combination therapies comprising an anti-CD38 antibody and cyclophosphamide for the treatment of CD38-positive hematological malignancies.

### BACKGROUND OF THE INVENTION

Multiple Myeloma (MM) is a B cell malignancy characterized by the latent accumulation of secretory plasma cells in bone marrow with a low proliferative index and an extended life span. The disease ultimately attacks bones and bone marrow, resulting in multiple tumors and lesions throughout the skeletal system. Approximately 1% of all cancers, and slightly more than 10% of all hematologic malignancies, can be attributed to MM. Incidence of MM increases in the aging population, with the median age at time of diagnosis being about 61 years.

Currently available therapies for MM include chemotherapy regimens, stem cell transplantation, THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid) and DARZALEX® (daratumumab). Current treatment protocols, which include a combination of chemotherapeutic agents such as vincristine, carmustine (BCNU), melphalan (Alkeran®), cyclophosphamide, doxorubicin (Adriamycin), and prednisone or dexamethasone, yield a complete remission rate of only about 5%, and median survival is approximately 36-48 months from the time of diagnosis. Recent advances using high dose chemotherapy followed by autologous bone marrow or peripheral blood mononuclear cell transplantation have increased the complete remission rate and remission duration. Nevertheless, overall survival has only been slightly prolonged, and no evidence for a cure has been obtained as yet. Ultimately, it it thought that all MM patients will relapse, even under maintenance therapy with interferon-alpha (IFN-α) alone or in combination with steroids.

Efficacy of the available drug treatment regimens for MM is limited by the low cell proliferation rate and development of drug resistance in up to 90% of patients. Chromosomal translocations, oncogene mutations, dysregulated signaling pathways such as anti-apoptotic and survival pathways, and bone marrow (BM) niche have been implicated to contribute to drug resistance in MM (for review, see Abdi et al., Oncotarget 4: 2186-2207, 2013). The BM niche is implicated in proliferation, survival, differentiation, migration, and drug resistance of the malignant plasma cells (Manier et al., J Biomed Biotechnol 2012; published online 2012 Oct. 3, doi:_10.1155/_2012/_157496).

### SUMMARY OF THE INVENTION

Provided herein are methods of treating a subject having a CD38-positive hematological malignancy, the methods comprising administering to the subject a therapeutically effective amount of an anti-CD38 antibody and cyclophosphamide for a time sufficient to treat the CD38-positive hematological malignancy. In some aspects of the methods, a therapeutically effective amount of dexamethasone, lenalidomide, bortezomib, or any combination thereof may also be administered to the subject.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 illustrates a flow cytometry analysis of CD38 expression on multiple myeloma (MM) cells (MM1S) 48 hours after treatment with cyclophosphamide (CTX) alone (CTX 2.5 μM, 5 μM, and 10 μM), or cyclophosphamide (10 μM) in combination with bortezomib (Bort; 1 nM) or lenalidomide (Len; 1 μM). The mean +/− SEM for two independent experiments is shown.

FIG. 2A illustrates MM1S cell surface expression of CD47 24 hours after treatment with lenalidomide (Len). Cells were treated with 0 μM, 0.01 μM, 0.1 μM, or 1 μM Len. CD47 mean fluorescent index (MFI) was measured.

FIG. 2B illustrates MM1S cell surface expression of CD47 24 hours after treatment with bortezomib (Bort). Cells were treated with 0 nM, 0.4 nM, 0.8 nM, or 1 nM Bort. CD47 mean fluorescent index (MFI) was measured.

FIG. 2C illustrates MM1S cell surface expression of CD47 24 hours after treatment with cyclophosphamide alone (CTX 2.5 μM, 5 μM, and 10 μM), or cyclophosphamide (10 μM) in combination with bortezomib (Bort; 1 nM) or lenalidomide (Len; 1μM). The mean +/− SEM for two independent experiments is shown.

FIG. 3A illustrates flow cytometry analysis of CD64 (FcyRI) expression on THP-1 macrophages after 48 hour incubation of the cells in conditioned media from untreated MM1S cells (CTX 0 μM) or MM1S cells treated with cyclophosphamide alone (CTX 2.5 μM, 5 μM, and 10 μM) or cyclophosphamide (10 μM) in combination with bortezomib (Bort; 1 nM) or lenalidomide (Len; 1 μM). The mean +/− SEM for two independent experiments is shown.

FIG. 3B illustrates flow cytometry analysis of CD32 (FcγRIIa/b) expression on THP-1 macrophages after 48 hour incubation of the cells in conditioned media from untreated MM1S cells (CTX 0 μM) or MM1S cells treated with cyclophosphamide alone (CTX 2.5 μM, 5 μM, and 10 μM) or cyclophosphamide (10 μM) in combination with bortezomib (Bort; 1 nM) or lenalidomide (Len; 1 μM). The mean +/− SEM for two independent experiments is shown.

FIG. 4A illustrates daratumumab-mediated MM1S tumor cell clearance in co-culture with THP-1 macrophages that were cultured 48 hours in conditioned media from untreated MM1S cells or MM1S cells treated with 0 μM, 0.01 μM, 0.1 μM, or 1 μM lenalidomide (Len) prior to start of the co-culture. % specific MM1S cell clearance was measured. Cell frequency was determined using the Accuri C6 (Right). MM1S cells were stained with calcein.

FIG. 4B illustrates daratumumab-mediated MM1S tumor cell clearance in co-culture with THP-1 macrophages that were cultured 48 hours in conditioned media from untreated MM1S cells or MM1S cells treated with 0 nM, 0.4 nM, or 0.8 nM bortezomib (Bort) prior to start of the co-culture. % specific MM1S cell clearance was measured. Cell frequency was determined using the Accuri C6 (Right). MM1S cells were stained with calcein.

FIG. 4C illustrates daratumumab-mediated MM1S tumor cell clearance in co-culture with THP-1 macrophages that were cultured 48 hours in conditioned media from untreated MM1S cells or MM1S cells treated with 0 μM, 2.5 μM, 5 μM, and 10 μM cyclophosphamide (CTX) or cyclophosphamide (10 μM) in combination with bortezomib (Bort; 1 nM) or lenalidomide (Len; 1 μM). Cell frequency was determined using the Accuri C6 (Right). MM1S cells were stained with calcein.

FIG. 5A illustrates effect of cytochalasin D (Cyto D) on daratumumab-mediated MM1S tumor cell clearance in co-culture with THP-1 macrophages that were cultured 48 hours in conditioned media from untreated MM1S cells or MM1S cells treated with 0, 0.01 μM, 0.1 μM or 1 μM lenalidomide (Len) prior to start of the co-culture. Cytochalasin D was either absent or added into co-cultures at 1 ?g/ml concentration.

FIG. 5B illustrates effect of cytochalasin D (Cyto D) on daratumumab-mediated MM1S tumor cell clearance in co-culture with THP-1 macrophages that were cultured 48 hours in conditioned media from untreated MM1S cells or MM1S cells treated with 0 or 10 μM cyclophosphamide, 10 μM cyclophosphamide and 1 nM boretezomib, or 10 μM cyclophosphamide and 1.0 μM lenalidomide prior to start of the co-culture. Cytochalasin D was either absent or added into co-cultures at 1 μg/ml concentration.

FIG. 6A illustrates a flow cytometry analysis of CD47 mean fluorescent expression (MFI) on CD45-CD19-CD138+ plasma cells in freshly isolated mononuclear cells (MNC) from peripheral blood (PB) of newly diagnosed MM patients (n=3) (pre-screened) prior to and 24 hours following cyclophosphamide treatment (150-300 mg/m²) (post-CTX).

FIG. 6B illustrates a flow cytometry analysis of CD47 mean fluorescent expression (MFI) on CD45-CD19-CD138+ plasma cells in freshly isolated mononuclear cells (MNC) from bone marrow (BM) of newly diagnosed MM patients (n=3) (pre-screened) prior to and 24 hours following cyclophosphamide treatment (150-300 mg/m²) (post-CTX). P=0.0555 between the two groups.

FIG. 7A illustrates a flow cytometry analysis of CD64 (FcγRI) expression on CD45+CD56−CD33+CX3CR1+CD14+ macrophages in freshly isolated MNC from PB of newly diagnosed MM patients (n=11) prior (pre-screened) to and 24 hours following cyclophosphamide treatment (150-300 mg/m²) (24hr post-CTX). The graph shows results from 11 patients in 11 independent experiments. *p=0.0329 at baseline vs. 24 hours post-treatment.

FIG. 7B illustrates a flow cytometry analysis of CD64 (FcγRI) expression on CD45+CD56−CD33+CX3CR1+CD14+ macrophages in freshly isolated MNC from BM of newly diagnosed MM patients (n=11) prior (pre-screened) to and 24 hours following cyclophosphamide treatment (150-300 mg/m²)) (24 hr post-CTX). The graph shows results from 11 patients in 11 independent experiments. *p=0.0018 at baseline vs 24 hours post-treatment.

FIG. 7C illustrates a flow cytometry analysis of CD32 (FcγRlIa/b) expression on CD45+CD56−CD33+CX3CR1+CD14+ macrophages in freshly isolated MNC from PB of newly diagnosed MM patients (n=11) prior to (pre-screened) and 24 hours following cyclophosphamide treatment (150-300 mg/m²) (24 hr post-CTX). The graphs shows rsults from 11 patients in 11 independent experiments.

FIG. 7D illustrates a flow cytometry analysis of CD32 (FcγRlIa/b) expression on CD45+CD56−CD33+CX3CR1+CD14+ macrophages in freshly isolated MNC from BM of newly diagnosed MM patients (n=11) prior to (pre-screened) and 24 hours following cyclophosphamide treatment (150-300 mg/m²) (24 hr post-CTX). The graphs shows rsults from 11 patients in 11 independent experiments. p=0.00371 at baseline vs 24 hours post-treatment.

FIG. 7E illustrates a flow cytometry analysis of SIRP1α expression on CD45+CD56−CD33+CX3CR1+CD14+ macrophages in freshly isolated MNC from PB of newly diagnosed MM patients (n=9) prior to (pre-screened) and 24 hours following cyclophosphamide treatment (150-300 mg/m²) (24 hr post-CTX). The graphs shows rsults from 9 patients in 9 independent experiments.

FIG. 7F illustrates a flow cytometry analysis of SIRP1α expression on CD45+CD56−CD33+CX3CR1+CD14+ macrophages in freshly isolated MNC from BM of newly diagnosed MM patients (n=9) prior to (pre-screened) and 24 hours following cyclophosphamide treatment (150-300 mg/m²) (24 hr post-CTX). The graphs shows rsults from 9 patients in 9 independent experiments.

FIG. 8A illustrates daratumumab-mediated MM1S tumor cell clearance in co-culture with freshly isolated MNC containing CD45+CD14+ PB macrophages of a newly diagnosed MM patient (n=1) prior to (pre-screened) and 24 hours following cyclophosphamide treatment (150-300 mg/m²) (24 hr post-CTX). MM1S were stained with CFSE and cells cultured for 12 hours. The % live MM1S cells relative to CD45+CD14+ macrophages were determined using the BD Canto II™ flow cytometer. Dara=daratumumab.

FIG. 8B illustrates daratumumab-mediated MM1S tumor cell clearance in co-culture with freshly isolated MNC containing CD45+CD14+ BM macrophages of a newly diagnosed MM patient (n=1) prior to (pre-screened) and 24 hours following cyclophosphamide treatment (150-300 mg/m²) (24 hr post-CTX). MM1S were stained with CFSE and cells cultured for 12hours. The % live MM1S cells relative to CD45+CD14+ macrophages were determined using the BD Canto II™ flow cytometer. Dara=daratumumab.

FIG. 8C. illustrates daratumumab-mediated ADCP of MM1S tumor cells in co-culture with freshly isolated MNC containing CD45+CD14+PB and BM macrophages of a newly diagnosed MM patient (n=1) prior to and 24 hours following cyclophosphamide treatment (150-300 mg/m²). MM1S were stained with CFSE then conditioned with Dara for 30 mins while macrophages were conditioned with or without 1 μg/ml cytochalasin D (CytoD) for 30 minutes prior to co-culture. Proportion of ADCP mediated clearance was calculated using the following formula: 100-100*(% live MM1S cells relative to CD45+CD14+ macrophages -CytoD/% live MM1S cells relative to CD45+CD14+ macrophages+CytoD).

### DETAILED DESCRIPTION OF THE INVENTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

“About” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of±10% or less, variations of±5% or less, variations of±1% or less, variations of±0.5% or less, or variations of±0.1% or less from the specified value.

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“CD38” refers to the human CD38 protein (UniProtKB accession no. P28907) (synonyms: ADP-ribosyl cyclase 1, cADPr hydrolase 1, cyclic ADP-ribose hydrolase 1). Human CD38 has an amino acid sequence as shown in SEQ ID NO: 1. CD38 is a single pass type II transmembrane protein with amino acid residues 1-21 representing the cytosolic domain, amino acid residues 22-42 representing the transmembrane domain, and residues 43-300 representing the extracellular domain. Anti-CD38 antibodies are described, for example, in Int'l Pat. Pub. No. WO2008/037257, Int'l Pat. Pub. No. WO2008/047242 and Int'l Pat. Pub. No. WO2007/042309, and are being evaluated in clinical settings for their efficacy in multiple myeloma and other hematological malignancies.

“Antibody,” is meant in a broad sense and includes immunoglobulin molecules including, monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies. Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full length antibody. Exemplary antibody fragments are heavy chain complementarity determining regions (HCDR) 1, 2, and 3, light chain complementarity determining regions (LCDR) 1, 2, and 3, a heavy chain variable region (VH), or a light chain variable region (VL). Antibody fragments include: a Fab fragment, a monovalent fragment consisting of the VL, VH, constant light (CL), and (constant heavy 1) CH1 domains; a F(ab)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; and a domain antibody (dAb) fragment (Ward et al., Nature 341: 544- 546, 1989), which consists of a VH domain or a VL domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

“Isolated antibody” refers to an antibody or antibody fragment that is substantially free of other antibodies having different antigenic specificities (e.g., an isolated anti-CD38 antibody is substantially free of antibodies that specifically bind antigens other than human CD38) or to a homogenous population of antibodies which have been substantially separated and/or purified away from other components of the system the antibody is produced in, such as a recombinant cell, as well as antibodies that have been subjected to at least one purification or isolation step An isolated anti-CD38 antibody, can have cross-reactivity to other antigens, such as orthologs of human CD38, such as Macaca fascicularis (cynomolgus) CD38.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132: 211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196: 901-17, 1987). Other terms include “IMGT-CDRs” (Lefranc et al., Dev Comparat Immunol 27: 55-77, 2003) and “Specificity Determining Residue Usage” (SDRU) (Almagro Mol Recognit 17: 132-43, 2004). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27: 55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the framework regions are derived from human immunoglobulin sequences. Humanized antibodies may include substitutions in the framework regions so that the framework may not be an exact copy of expressed human immunoglobulin or germline gene sequences. If the antibody contains a constant region, the constant region is also derived from sequences of human origin. “Derived from,” as used in the context of humanized antibodies, means that the region in question is at least 80% homologous in sequence to the corresponding region of the immunoglobulin from the species in which it is based.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. “Human antibody” may contain amino acid differences when compared to the human germline or rearranged immunoglobulin sequences due to, for example, naturally occurring somatic mutations or intentional introduction of substitutions in the framework or antigen binding sites. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene. In some cases, a “human antibody” may contain consensus framework sequences derived from human framework sequence analyses, for example as described in Knappik et al., J Mol Biol 296: 57-86, 2000, or synthetic HCDR3 incorporated into human immunoglobulin gene libraries displayed on phage, as described in, for example, Shi et al., J Mol Biol 397: 385-96, 2010 and Int'l Pat. Pub. No. WO2009/085462. Antibodies in which antigen binding sites are derived from a non-human species are not included in the definition of “human antibody”.

“Recombinant antibody” includes all antibodies that are prepared, expressed, created, or isolated by recombinant means, such as: antibodies isolated from an animal (e g , a mouse) that is transgenic or transchromosomal for human immunoglobulin genes or a hybridoma prepared therefrom (described further below); antibodies isolated from a host cell transformed to express the antibody; antibodies isolated from a recombinant, combinatorial antibody library; and antibodies prepared, expressed, created, or isolated by any other means that involve splicing of human immunoglobulin gene sequences to other DNA sequences, or antibodies that are generated in vitro using Fab arm exchange.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well known alterations such as removal of C-terminal lysine from the antibody heavy chain Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by the CD38-positive hematological malignancy. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed combination therapy effective, at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

“Inhibit growth” (e.g., referring to cells, such as tumor cells) refers to a measurable decrease in in vitro or in vivo cell growth upon contact with a therapeutic or the combination therapy when compared to the growth of the same cells in the absence of the combination therapy. Inhibition of growth of a cell in vitro or in vivo may be at least about 10%, about 20%, about 30%, about 40%, about 50%, about 60%, about 70%, about 80%, about 90%, about 99%, or about 100%. Inhibition of cell growth can occur by a variety of mechanisms, for example by ADCC, apoptosis, necrosis, or by inhibition of cell proliferation.

“Subject” includes any human or nonhuman animal “Nonhuman animal” includes all vertebrates, e g , mammals and non-mammals, such as nonhuman primates, sheep, dogs, cats, horses, cows, chickens, amphibians, reptiles, etc. The terms “subject” and “patient” can be used interchangeably herein.

The following abbreviations are used throughout the disclosure: acute lymphoblastic leukemia (ALL), acute myeloid leukemia (AML), antibody-dependent cellular cytotoxicity (ADCC), antibody-dependent cellular phagocytosis (ADCP), bortezomib (Bort); Burkitt's lymphoma (BL), chronic lymphocytic leukemia (CLL), complement-dependent cytotoxicity (CDC), complementarity determining region (CDR), constant light (CL), (constant heavy 1) CH1 domains, cyclophosphamide (CTX); daratumumab (DARA); diffuse large B-cell lymphoma (DLBCL), follicular lymphoma (FL), heavy chain CDR (HCDR), heavy chain variable region (VH), Hodgkin's lymphoma (HL), lenalidomide (LEN); light chain CDR (LCDR), light chain variable region (VL), multiple myeloma (MM), mantle-cell lymphoma (MCL); non-Hodgkin's lymphoma (NHL); bone marrow (BM); peripheral blood (PB); mononuclear cell (MC).

Provided here are methods of treating a subject having a CD38-positive hematological malignancy, comprising administering to the subject a therapeutically effective amount of an anti-CD38 antibody and cyclophosphamide for a time sufficient to treat the CD38-positive hematological malignancy.

“CD38-positive hematological malignancy” refers to a hematological malignancy characterized by the presence of tumor cells expressing CD38, including leukemias, lymphomas, and myeloma. Exemplary CD38-positive hematological malignancies are precursor B-cell lymphoblastic leukemia/lymphoma, B-cell non-Hodgkin's lymphoma, acute promyelocytic leukemia, acute lymphoblastic leukemia and mature B-cell neoplasms, such as B-cell chronic lymphocytic leukemia(CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL) (including low-grade, intermediate- grade and high-grade FL), cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma (MM), plasma cell leukemia, post-transplant lymphoproliferative disorder, Waldenstrom's macroglobulinemia, plasma cell leukemias, anaplastic large-cell lymphoma (ALCL), T-cell acute lymphocytic leukemia, primary systemic amyloidosis, mantle-cell lymphoma, pro-lymphocytic/myelocytic leukemia, acute myeloid leukemia (AML) (including acute promyelocytic leukemia), chronic myeloid leukemia (CML), large granular lymphocytic (LGL) leukemia, NK-cell leukemia, T-cell prolymphocytic leukemia, T-cell large granular lymphocytic leukemia, aggressive NK cell leukemia, adult T-cell leukemia/lymphoma, nasal type extranodal NK/T cell lymphoma, 78 enteropathy-type T-cell lymphoma, hepatosplenic T-cell lymphoma, subcutaneous panniculitis-like T-cell lymphoma, blastic NK cell lymphoma, Mycosis Fungoides/Sezary Syndrome, primary cutaneous CD38 positive T-cell lymphoproliferative disorders (primary cutaneous anaplastic large cell lymphoma C-ALCL, lymphomatoid papulosis, borderline lesions), angioimmunoblastic T-cell lymphoma, peripheral T-cell lymphoma unspecified, and anaplastic large cell lymphoma.

Examples of B-cell non-Hodgkin's lymphomas include lymphomatoid granulomatosis, primary effusion lymphoma, intravascular large B-cell lymphoma, mediastinal large B-cell lymphoma, heavy chain diseases (including ?, ?, and a disease), lymphomas induced by therapy with immunosuppressive agents, such as cyclosporine-induced lymphoma, and methotrexate-induced lymphoma.

In some embodiments, the CD38-positive hematological malignancy is multiple myeloma (MM). In some embodiments, the CD38-positive hematological malignancy is acute lymphoblastic leukemia (ALL). In some embodiments, the CD38-positive hematological malignancy is non-Hodgkin's lymphoma (NHL). In some embodiments, the CD38-positive hematological malignancy is diffuse large B-cell lymphoma (DLBCL). In some embodiments, the CD38-positive hematological malignancy is Burkitt's lymphoma (BL). In some embodiments, the CD38-positive hematological malignancy is follicular lymphoma (FL). In some embodiments, the CD38-positive hematological malignancy is mantle-cell lymphoma (MCL). In some embodiments, the CD38-positive hematological malignancy is acute myeloid leukemia (AML). In some embodiments, the CD38-positive hematological malignancy is chronic lymphocytic leukemia (CLL). In some embodiments, the CD38-positive hematological malignancy is any combination of the above CD38-positive hematological malignancies. Thus, disclosed herein are methods of treating a subject having multiple myeloma, comprising administering to the subject a therapeutically effective amount of an anti-CD38 antibody and cyclophosphamide for a time sufficient to treat the multiple myeloma.

In some embodiments, the CD38-positive hematological malignancy is not light chain amyloidosis (AL). In such embodiments, the methods of treating a subject having a CD38-positive hematological malignancy comprise administering to the subject a therapeutically effective amount of an anti-CD38 antibody and cyclophosphamide for a time sufficient to treat the CD38-positive hematological malignancy, wherein the CD38-positive hematological malignancy is not light chain amyloidosis.

Any anti-CD38 antibody may be used in the disclosed methods. For example, the variable regions of the anti-CD38 antibodies may be obtained from existing anti-CD38 antibodies and optionally cloned as full length antibodies using standard methods. Exemplary antibody variable regions that bind CD38 that may be used are described in Int'l Pat. Pub. Nos. WO2005/103083, WO2006/125640, WO2007/042309, WO2008/047242, WO2012/092612, WO2006/099875, and WO2011/154453A1.

The anti-CD38 antibody can bind to a region of human CD38 comprising SKRNIQFSCKNIYR (SEQ ID NO: 2) and a region of human CD38 comprising EKVQTLEAWVIHGG (SEQ ID NO: 3). An anti-CD38 antibody binds to a region of human CD38 comprising SEQ ID NO: 2 and a region of human CD38 comprising SEQ ID NO: 3 when the antibody binds at least 1, 2, 3, 4, 5, 6, 7, 8, 9, 10, 11, 12, 13, or 14 residues within SEQ ID NO: 2 and at least 1, 2, 3, 4, 5, 6, 7, 8, 9, 10, 11, 12, 13, or 14 residues within SEQ ID NO: 3. In some embodiments, the anti-CD38 antibody binds at least one amino acid in a region of human CD38 comprising SEQ ID NO: 2 and at least one amino acid in a region of human CD38 comprising SEQ ID NO: 3. In some embodiments, the anti-CD38 antibody binds at least two amino acids in a region of human CD38 comprising SEQ ID NO: 2 and at least two amino acids in a region of human CD38 comprising SEQ ID NO: 3. In some embodiments, the anti-CD38 antibody binds at least three amino acids in a region of human CD38 comprising SEQ ID NO: 2 and at least three amino acids in a region of human CD38 comprising SEQ ID NO: 3. Antibodies binding to a region of human CD38 comprising SEQ ID NO: 2 and a region of human CD38 comprising SEQ ID NO: 3 may be generated, for example, by immunizing mice with peptides having amino acid sequences comprising SEQ ID NOs: 2 and 3 using standard methods and as described herein, and characterizing the obtained antibodies for binding to the peptides using, for example, ELISA or mutagenesis studies.

An exemplary anti-CD38 antibody that binds to a region of human CD38 comprising SEQ ID NO: 2 and a region of human CD38 comprising SEQ ID NO: 3 is DARZALEXTM (daratumumab), which comprises: a heavy chain complementarity determining region (CDR) 1, a heavy chain CDR2, and a heavy chain CDR3 of SEQ ID NOs: 6, 7, and 8, respectively, and a light chain complementarity determining region (CDR) 1, a light chain CDR2, and a light chain CDR3 of SEQ ID NOs: 9, 10, and 11, respectively, a heavy chain variable region (VH) of SEQ ID NO: 4 and a light chain variable region (VL) of SEQ ID NO: 5; and a heavy chain amino acid sequence of SEQ ID NO: 12 and light chain amino acid sequence of SEQ ID NO: 13.

The anti-CD38 antibody can comprise a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11. The anti-CD38 antibody can comprise a VH comprising an amino acid sequence that is 95%, 96%, 97%, 98%, 99%, or 100% identical to that of SEQ ID NO: 4 and a VL comprising an amino acid sequence that is 95%, 96%, 97%, 98%, 99%, or 100% identical to that of SEQ ID NO: 5. In some embodiments, the anti-CD38 antibody can comprise a heavy chain variable region (VH) comprising the amino acid sequence of SEQ ID NO: 4 and a light chain variable region (VL) comprising the amino acid sequence of SEQ ID NO: 5. The anti-CD38 antibody can comprise a heavy chain comprising the amino acid sequence of SEQ ID NO: 12 and a light chain comprising the amino acid sequence of SEQ ID NO: 13.

The anti-CD38 antibody can comprise a heavy chain CDR1, a heavy chain CDR2, and a heavy chain CDR3 of a VH comprising the amino acid sequence of SEQ ID NO: 14, and a light chain CDR1, a light chain CDR2, and a light chain CDR3 of a VL comprising the amino acid sequence of SEQ ID NOs:15. The anti-CD38 antibody can comprise a VH comprising an amino acid sequence that is 95%, 96%, 97%, 98%, 99%, or 100% identical to that of SEQ ID NO: 15 and a VL comprising an amino acid sequence that is 95%, 96%, 97%, 98%, 99%, or 100% identical to that of SEQ ID NO: 15. In some embodiments, the anti-CD38 antibody can comprise a VH comprising the amino acid sequence of SEQ ID NO: 14 and a VL comprising the amino acid sequence of SEQ ID NOs:15. In some embodiments, for example, the anti-CD38 antibody can comprise mAb003 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 16, and a light chain CDR1, a light chain CDR2, and a light chain CDR3 of a VL comprising the amino acid sequence of SEQ ID NO: 17. The anti-CD38 antibody can comprise a VH comprising an amino acid sequence that is 95%, 96%, 97%, 98%, 99%, or 100% identical to that of SEQ ID NO: 16 and a VL comprising an amino acid sequence that is 95%, 96%, 97%, 98%, 99%, or 100% identical to that of SEQ ID NO: 17. In some embodiments, the anti-CD38 antibody can comprise a VH comprising the amino acid sequence of SEQ ID NO: 16 and a VL comprising the amino acid sequence of SEQ ID NO: 17. In some embodiments, for example, the anti-CD38 antibody can comprise mAb024 (described in U.S. Pat. No. 7,829,693, incorporated herein by reference).

The anti-CD38 antibody can comprise a heavy chain CDR1, a heavy chain CDR2, and a heavy chain CDR3 of a VH comprising the amino acid sequence of SEQ ID NO: 18, and a light chain CDR1, a light chain CDR2, and a light chain CDR3 of a VL comprising the amino acid sequence of SEQ ID NO: 19. The anti-CD38 antibody can comprise a VH comprising an amino acid sequence that is 95%, 96%, 97%, 98%, 99%, or 100% identical to that of SEQ ID NO: 18 and a VL comprising an amino acid sequence that is 95%, 96%, 97%, 98%, 99%, or 100% identical to that of SEQ ID NO: 19. In some embodiments, the anti-CD38 antibody can comprise a VH comprising the amino acid sequence of SEQ ID NO: 18 and a VL comprising the amino acid sequence of SEQ ID NO: 19. In some embodiments, for example, the anti-CD38 antibody can comprise MOR-202 (MOR-03087) (described in US. Pat. No. 8,088,896, incorporated herein by reference).

The anti-CD38 antibody can comprise a heavy chain CDR1, a heavy chain CDR2, and a heavy chain CDR3 of a VH comprising the amino acid sequence of SEQ ID NO: 20, and a light chain CDR1, a light chain CDR2, and a light chain CDR3 of a VL comprising the amino acid sequence of SEQ ID NO: 21. The anti-CD38 antibody can comprise a VH comprising an amino acid sequence that is 95%, 96%, 97%, 98%, 99%, or 100% identical to that of SEQ ID NO: 20 and a VL comprising an amino acid sequence that is 95%, 96%, 97%, 98%, 99%, or 100% identical to that of SEQ ID NO: 21. In some embodiments, the anti-CD38 antibody can comprise a VH comprising the amino acid sequence of SEQ ID NO: 20 and a VL comprising the amino acid sequence of SEQ ID NO: 21. In some embodiments, for example, the anti-CD38 antibody can comprise isatuximab (described in U.S. Pat. No. 8,153,765, incorporated herein by reference). In some aspects, the VH and the VL of isatuximab may be expressed as IgG1/κ.

Antibodies that are substantially identical to those disclosed herein may be used in the disclosed methods. The term “substantially identical” means that the antibody heavy chain or light chain amino acid sequences are identical, or have “insubstantial differences,” compared to the antibody disclosed herein. Insubstantial differences are substitutions of 1, 2, 3, 4, 5, 6, 7, 8, 9, 10, 11, 12, 13, 14, or 15 amino acids in an antibody heavy chain or light chain that do not adversely affect antibody properties. Antibody sequences can be compared, for example, by pairwise alignments using the default settings of the AlignX module of Vector NTI v.9.0.0 (Invitrogen, Carlsbad, Calif.). The protein sequences of the disclosed antibodies can be used as a query sequence to perform a search against public or patent databases to, for example, identify related sequences. Exemplary programs used to perform such searches are the XBLAST or BLASTP programs (http_//www_ncbi_nlm/nih_gov), or the GenomeQuest™ (GenomeQuest, Westborough, Mass.) suite using the default settings. Antibodies that are substantially identical to the disclosed antibodies can be generated, for example, by making conservative modifications to the amino acid sequences of the disclosed antibodies. “Conservative modifications” refer to amino acid modifications that do not significantly affect or alter the binding characteristics of the antibody containing the amino acid sequences. Conservative modifications include amino acid substitutions, additions, and deletions. “Conservative substitutions” are those in which the amino acid is replaced with an amino acid residue having a similar side chain The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), an amide (e.g., asparagine, glutamine), beta-branched side chains (e.g., threonine, valine, isoleucine), and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643: 55-67; Sasaki et al., (1988) Adv Biophys 35: 1-24). Exemplary substitutions that can be made to the anti-CD38 antibodies used in the disclosed methods include, for example, conservative substitutions with an amino acid having similar charge, hydrophobic, or stereochemical characteristics. Conservative substitutions may also be made to improve antibody properties, including stability or affinity, or to improve antibody effector functions. 1, 2, 3, 4, 5, 6, 7, 8, 9, 10, 11, 12, 13, 14, or 15 amino acid substitutions may be made, for example, to the heavy and/or the light chain of the anti-CD38 antibody. Furthermore, any native residue in the heavy and/or light chain may also be substituted with alanine, as has been previously described for alanine scanning mutagenesis (MacLennan et al., Acta Physiol Scand Suppl 643: 55-67, 1998; Sasaki et al., Adv Biophys 35: 1-24, 1998). Suitable amino acid substitutions may be determined by those skilled in the art at the time such substitutions are desired Amino acid substitutions may be performed, for example, by PCR mutagenesis (as disclosed in U.S. Pat. No. 4,683,195). Libraries of variants may be generated using well-known methods; for example using random (NNK) or non-random codons (for example DVK codons) which encode 11 amino acids (Ala, Cys, Asp, Glu, Gly, Lys, Asn, Arg, Ser, Tyr, Trp) and screening the libraries for variants with desired properties. The generated variants may be tested for their binding to CD38 and their ability to induce ADCC using methods described herein.

The anti-CD38 antibody can be of the IgG1, IgG2, IgG3, or IgG4 isotype. In some embodiments, the anti-CD38 antibody is of the IgG1 isotype. In some embodiments, the anti-CD38 antibody is of the IgG2 isotype. In some embodiments, the anti-CD38 antibody is of the IgG3 isotype. In some embodiments, the anti-CD38 antibody is of the IgG4 isotype.

Anti-CD38 antibodies used in the disclosed methods may also be selected de novo from, for example, a phage display library, where the phage is engineered to express human immunoglobulins or portions thereof such as Fabs, single chain antibodies (scFv), or unpaired or paired antibody variable regions (Knappik et al., J Mol Biol 296: 57-86, 2000; Krebs et al., J Immunol Meth 254: 67-84, 2001; Vaughan et al., Nature Biotechnology 14: 309-314, 1996; Sheets et al., PITAS (USA) 95: 6157-6162, 1998; Hoogenboom and Winter, J Mol Biol 227: 381, 1991; Marks et al., J Mol Biol 222: 581, 1991). CD38 binding variable domains may be isolated, for example, from phage display libraries expressing antibody heavy and light chain variable regions as fusion proteins with bacteriophage pIX coat protein as described in Shi et al (2010) J. Mol. Biol. 397: 385-96 and Int'l Pat. Pub. No. WO2009/085462. The antibody libraries may be screened for binding to human CD38 extracellular domain and the obtained positive clones may be further characterized and the Fabs isolated from the clone lysates, and subsequently cloned as full length antibodies. Such phage display methods for isolating human antibodies are established in the art. See for example: U.S. Pat. No. 5,223,409; U.S. Pat. No. 5,403,484; U.S. Pat. No. 5,571,698; U.S. Pat. No. 5,427,908; U.S. Pat. No. 5,580,717; U.S. Pat. No. 5,969,108; U.S. Pat. No. 6,172,197; U.S. Pat. No. 5,885,793; U.S. Pat. No. 6,521,404; U.S. Pat. No. 6,544,731; U.S. Pat. No. 6,555,313; U.S. Pat. No. 6,582,915; and U.S. Pat. No. 6,593,081.

Also disclosed are methods of treating a subject having a CD38-positive hematological malignancy, comprising administering to the subject a therapeutically effective amount of: an anti-CD38 antibody that competes for binding to CD38 with a reference antibody and cyclophosphamide,

wherein the reference antibody comprises:
a) a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising an amino acid sequence of SEQ ID NO: 11;
b) a VH comprising the amino acid sequence of SEQ ID NO: 4 and a VL comprising the amino acid sequence of SEQ ID NO: 5;
c) a heavy chain comprising the amino acid sequence of SEQ ID NO: 12 and a light chain comprising the amino acid sequence of SEQ ID NO: 13;
d) DARZALEX® (daratumumab);
e) a heavy chain CDR1, a heavy chain CDR2, and a heavy chain CDR3 of a VH comprising the amino acid sequence of SEQ ID NO: 14, and a light chain CDR1, a light chain CDR2, and a light chain CDR3 of a VL comprising the amino acid sequence of SEQ ID NO: 15;
f) a VH comprising the amino acid sequence of SEQ ID NO: 14 and a VL comprising the amino acid sequence of SEQ ID NO: 15;
g) mAb003;
h) a heavy chain CDR1, a heavy chain CDR2, and a heavy chain CDR3 of a VH comprising the amino acid sequence of SEQ ID NO: 16, and a light chain CDR1, a light chain CDR2, and a light chain CDR3 of a VL comprising the amino acid sequence of SEQ ID NO: 17;
i) a VH comprising the amino acid sequence of SEQ ID NO: 16 and a VL comprising the amino acid sequence of SEQ ID NO: 17;
j) mAb024;
k) a heavy chain CDR1, a heavy chain CDR2, and a heavy chain CDR3 of a VH comprising the amino acid sequence of SEQ ID NO: 18, and a light chain CDR1, a light chain CDR2, and a light chain CDR3 of a VL comprising the amino acid sequence of SEQ ID NO: 19;
l) a VH comprising the amino acid sequence of SEQ ID NO: 18 and a VL comprising the amino acid sequence of SEQ ID NO: 19;
m) MOR-202 (MOR-03087);
n) a heavy chain CDR1, a heavy chain CDR2, and a heavy chain CDR3 of a VH comprising the amino acid sequence of SEQ ID NO: 20, and a light chain CDR1, a light chain CDR2, and a light chain CDR3 of a VL comprising the amino acid sequence of SEQ ID NO: 21;
o) a VH comprising the amino acid sequence of SEQ ID NO: 20 and a VL comprising the amino acid sequence of SEQ ID NO: 21;
p) isatuximab; or
q) any combination of a) to p).

In some embodiments, the CD38-positive hematological malignancy is multiple myeloma, and the methods comprise administering to the subject a therapeutically effective amount of:

an anti-CD38 antibody that competes for binding to CD38 with a reference antibody and cyclophosphamide,
wherein the reference antibody comprises any one of a) to q) above.

Antibodies may be evaluated for their competition with a reference antibody (such as references antibodies a) to q) above) for binding to CD38 using well known in vitro methods. In an exemplary method, CHO cells recombinantly expressing CD38 may be incubated with unlabeled reference antibody for 15 min at 4? C, followed by incubation with an excess of fluorescently labeled test antibody for 45 min at 4° C. After washing in PBS/BSA, fluorescence may be measured by flow cytometry using standard methods. In another exemplary method, an extracellular portion of human CD38 may be coated on the surface of an ELISA plate. Excess unlabeled reference antibody may be added for about 15 minutes and subsequently biotinylated test antibodies may be added. After washes in PBS/Tween, binding of the test biotinylated antibody may be detected using horseradish peroxidase (HRP)-conjugated streptavidin and the signal detected using standard methods. In the competition assays, the reference antibody may be labeled and the test antibody may be unlabeled. The test antibody competes with the reference antibody when the reference antibody inhibits binding of the test antibody, or the test antibody inhibits binding of the reference antibody, by at least about 90%, 95%, or 100%. The epitope of the test antibody may further be defined, for example, by peptide mapping or hydrogen/deuterium protection assays using known methods, or by crystal structure determination.

The anti-CD38 antibody can induce killing of CD38-expressing cells by antibody-dependent cellular cytotoxicity (ADCC), antibody-dependent cellular phagocytosis (ADCP), complement-dependent cytotoxicity (CDC), or apoptosis. In some embodiments, the anti-CD38 antibody induces killing of CD38-expressing cells by ADCC. In some embodiments, the anti-CD38 antibody induces killing of CD38-expressing cells by ADCP. In some embodiments, the anti-CD38 antibody induces killing of CD38-expressing cells by CDC. In some embodiments, the anti-CD38 antibody induces killing of CD38-expressing cells by apoptosis. In some embodiments, the anti-CD38 antibody induces killing of CD38-expressing cells by any combination of ADCC, ADCP, CDC, and apoptosis.

“Antibody-dependent cellular cytotoxicity,” “antibody-dependent cell-mediated cytotoxicity” or “ADCC” is a mechanism for inducing cell death that depends upon the interaction of antibody-coated target cells with effector cells possessing lytic activity, such as natural killer (NK) cells, monocytes, macrophages, and neutrophils via Fc gamma receptors (FcγR) expressed on effector cells. For example, NK cells express FcγRllla, whereas monocytes express FcγRI, FcγRII, and FcγRllla. Death of the antibody-coated target cell, such as CD38-expressing MM cell, occurs as a result of effector cell activity through the secretion of membrane pore-forming proteins and proteases. To assess ADCC activity of an anti-CD38 antibody, the antibody may be added to CD38-expressing cells in combination with immune effector cells, which may be activated by the antigen/antibody complexes resulting in cytolysis of the target cell. Cytolysis may be detected by the release of a label (e.g., radioactive substrates, fluorescent dyes, or natural intracellular proteins) from the lysed cells. Exemplary effector cells for such assays include peripheral blood mononuclear cells (PBMC) and NK cells. Multiple myeloma cell lines or primary MM cells that express CD38 may be used as target cells. In an exemplary assay, MM cell lines engineered to express luciferase are incubated with anti-CD38 antibodies. Freshly isolated PBMC effector cells are added at a target:effector cell ratio of 40:1. 4 hours after addition of PBMC, luciferin is added and the resulting bioluminescent signal emitted from surviving MM cells can be determined within 20 minutes using a luminometer (SpectraMax, Molecular Devices), and the percentage ADCC of MM cells can calculated using the formula: % ADCC=1−(mean bioluminescent signal in the absence of PBMCs/mean bioluminescent signal in the presence of PBMCs)×100%. Anti-CD38 antibodies used in the disclosed methods can induce ADCC by about 10%, 15%, 20%, 25%, 30%, 35%, 40%, 45%, 50%, 55%, 60%, 65%, 70%, 80%, 90%, or 100%.

“Complement-dependent cytotoxicity,” or “CDC,” refers to a mechanism for inducing cell death in which an Fc effector domain of a target-bound antibody binds and activates complement component Clq, which in turn activates the complement cascade leading to target cell death. Activation of complement may also result in deposition of complement components on the target cell surface that facilitate ADCC by binding complement receptors (e.g., CR3) on leukocytes. In an exemplary assay, primary BM-MNC cells isolated from a patient with a B-cell malignancy may be treated with an anti-CD38 antibody and complement derived from 10% pooled human serum for 1 hour at a concentration of 0.3-10 μg/ml, and the survival of primary CD38+ MM cells may be determined by flow cytometry using techniques described in van der Veer et al., Haematologica 96: 284-290, 2011; van der Veer et al., Blood Cancer J 1(10): e41, 2011. The percentage of MM cell lysis may be determined relative to an isotype control as described herein. Anti-CD38 antibodies used in the disclosed methods may induce CDC by about 10%, 15%, 20%, 25%, 30%, 35%, 40%, 45%, 50%, 55%, 60%, 65%, 70%, 75%, 80%, 85%, 90%, 95%, or 100%.

“Antibody-dependent cellular phagocytosis,” or “ADCP,” refers to a mechanism of elimination of antibody-coated target cells by internalization by phagocytic cells, such as macrophages or dendritic cells. ADCP may be evaluated by using monocyte-derived macrophages as effector cells and Daudi cells (ATCC® CCL-213™) or B cell leukemia or lymphoma tumor cells expressing CD38 as target cells engineered to express GFP or other labeled molecules. Effector:target cell ratio may be, for example, 4:1. Effector cells may be incubated with target cells for 4 hours with or without anti-CD38 antibody. After incubation, cells may be detached using accutase. Macrophages may be identified with anti-CD11b and anti-CD14 antibodies coupled to a fluorescent label, and percent phagocytosis may be determined based on % GFP fluorescence in the CD11+CD14+ macrophages using standard methods. Anti-CD38 antibodies used in the disclosed methods may induce ADCP by about 10%, 15%, 20%, 25%, 30%, 35%, 40%, 45%, 50%, 55%, 60%, 65%, 70%, 75%, 80%, 85%, 90%, 95% or 100% .

The Fc portion of the anti-CD38 antibody may mediate antibody effector functions such as antibody-dependent cell-mediated cytotoxicity (ADCC), antibody-dependent cellular phagocytosis (ADCP), or complement dependent cytotoxicity (CDC). Such functions may be mediated by binding of an Fc effector domain(s) to an Fc receptor on an immune cell with phagocytic or lytic activity, or by binding of an Fc effector domain(s) to components of the complement system. Typically, the effect(s) mediated by the Fc-binding cells or complement components result in inhibition and/or depletion of target cells, e.g., CD38-expressing cells. Human IgG isotypes IgG1, IgG2, IgG3, and IgG4 exhibit differential capacity for effector functions. ADCC may be mediated by IgG1 and IgG3, ADCP may be mediated by IgG1, IgG2, IgG3, and IgG4, and CDC may be mediated by IgG1 and IgG3.

ADCC elicited by the anti-CD38 antibodies may be enhanced by certain substitutions in the antibody Fc region. In some embodiments, the anti-CD38 antibodies comprise a substitution in the Fc region at amino acid position 256, 290, 298, 312, 356, 330, 333, 334, 360, 378, 430, or any combination thereof, wherein the residue numbering is according to the EU index (substitutions described in U.S. Pat. No. 6,737,056).

ADCC elicited by the anti-CD38 antibodies can also be enhanced by engineering an antibody oligosaccharide component. Human IgG1 or IgG3 are N-glycosylated at Asn297 with the majority of the glycans in the biantennary G0, G0F, G1, G1F, G2, or G2F forms. Antibodies produced by non-engineered CHO cells typically have a glycan fucose content (i.e. the amount of the fucose monosaccharide within the sugar chain at Asn297) of about at least 85%. The removal of the core fucose from the biantennary complex-type oligosaccharides attached to the Fc regions enhances the ADCC of antibodies via improved FcyRllla binding without altering antigen binding or CDC activity. Such modified antibodies can be achieved using different methods reported to lead to the successful expression of relatively high defucosylated antibodies bearing the biantennary complex-type of Fc oligosaccharides such as: control of culture osmolality (Konno et al., Cytotechnology 64:249-65, 2012); application of a variant CHO line Lec13 as the host cell line (Shields et al., J Biol Chem 277: 26733-26740, 2002); application of a variant CHO line EB66 as the host cell line (Olivier et al., MAbs 2(4), 2010; Epub ahead of print; PMID:20562582); application of a rat hybridoma cell line YB2/0 as the host cell line (Shinkawa et al., J Biol Chem 278: 3466-3473, 2003); introduction of small interfering RNA specifically against the ?1,6-fucosyltrasferase (FUT8) gene (Mori et al., Biotechnol Bioeng 88: 901-908, 2004); or coexpression of β-1,4-N-acetylglucosaminyltransferase III and Golgi a-mannosidase II or a potent alpha-mannosidase I inhibitor, such as kifunensine (Ferrara et al., J Biol Chem 281: 5032-5036, 2006, Ferrara et al., Biotechnol Bioeng 93: 851-861, 2006; Xhou et al., Biotechnol Bioeng 99: 652-65, 2008).

In some embodiments, the anti-CD38 antibody can have a biantennary glycan structure with fucose content between about 0% to about 15%, for example 15%, 14%, 13%, 12%, 11%, 10%, 9%, 8%, 7%, 6%, 5%, 4%, 3%, 2%, 1%, or 0%. In some embodiments, the anti-CD38 antibody can have a biantennary glycan structure with fucose content of about 50%, 45%, 40%, 35%, 30%, 25%, 20%, 15%, 14%, 13%, 12%, 11%, 10%, 9%, 8%, 7%, 6%, 5%, 4%, 3%, 2%, 1%, or 0%. Substitutions in the Fc region and reduced fucose content may enhance the ADCC activity of the anti-CD38 antibody.

Fucose content may be characterized and quantified by multiple methods, for example: 1) using MALDI-TOF of N-glycosidase F treated sample (e.g. complex, hybrid, and oligo- and high-mannose structures) as described in Int'l Pat. Pub. No. WO2008/0775462); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the antibody to constituent peptides by enzymatic digestion (e.g., trypsin or endopeptidase Lys-C), and subsequent separation, detection, and quantitation by HPLC-MS (UPLC-MS); and 5) separation of the antibody oligosaccharides from the antibody protein by specific enzymatic deglycosylation with PNGase F at Asn297. The oligosaccharides thus released can be labeled with a fluorophore, separated, and identified by various complementary techniques which allow: fine characterization of the glycan structures by matrix-assisted laser desorption ionization (MALDI) mass spectrometry by comparison of the experimental masses with the theoretical masses; determination of the degree of sialylation by ion exchange HPLC (GlycoSep C); separation and quantification of the oligosacharride forms according to hydrophilicity criteria by normal-phase HPLC (GlycoSep N); and separation and quantification of the oligosaccharides by high performance capillary electrophoresis-laser induced fluorescence (HPCE-LIF).

The anti-CD38 antibody may bind human CD38 with a range of affinities (K_{D}). For example, the anti-CD38 antibody can bind CD38 with a KD equal to or less than about 1×10⁻⁸ M, for example 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, 5×10⁻¹⁵ M, or any range or value therein, as determined by surface plasmon resonance or the Kinexa method, as practiced by those of skill in the art. In some embodiments, the anti-CD38 antibody can bind to CD38 with an affinity of equal to or less than 1×10⁻⁸ M. In some embodiments, the anti-CD38 antibody can bind to CD38 with an affinity of equal to or less than 1×10⁻⁹ M.

Antibody affinity can be measured using KinExA instrumentation, ELISA, or competitive binding assays known to those skilled in the art. The measured affinity of a particular antibody/CD38 interaction may vary if measured under different conditions (e.g., osmolarity, pH). Thus, measurements of affinity and other binding parameters (e.g., KD, Kon, Koff) are typically made with standardized conditions and a standardized buffer. Those skilled in the art will appreciate that the internal error for affinity measurements for example using Biacore 3000 or ProteOn (measured as standard deviation, SD) may typically be within 5-33% for measurements within the typical limits of detection. Therefore the term “about” in the context of K_{D} reflects the typical standard deviation in the assay. For example, the typical SD for a K_{D} of 1×10⁻⁹ M is up to+0.33×10⁻⁹ M.

In some embodiments, the antibody that specifically binds CD38 is a non-agonistic antibody. A non-agonistic antibody that specifically binds CD38 refers to an antibody which upon binding to CD38 does not induce significant proliferation of a sample of peripheral blood mononuclear cells in vitro when compared to the proliferation induced by an isotype control antibody or medium alone.

In some embodiments, the non-agonistic antibody that specifically binds CD38 induces proliferation of peripheral blood mononuclear cells (PBMCs) in a statistically insignificant manner PBMC proliferation may be assessed by isolating PBMCs from healthy donors and culturing the cells at 1×10⁵ cells/well in flat bottom 96-well plates in the presence or absence of a test antibody in 200 μl RPMI. After four day incubation at 37° C., 30 μl 3H-thymidine (16.7 μCi/ml) may be added, and culture may be continued overnight. 3H-thymidine incorporation may be assessed using a Packard Cobra gamma counter (Packard Instruments, Meriden, DT, USA), according to the manufacturer's instructions. Data may be calculated as the mean cpm (±SEM) of PBMCs obtained from several donors. Statistical significance or insignificance between samples cultured in the presence or absence of the test antibody is calculated using standard methods.

The anti-CD38 antibodies may be provided in suitable pharmaceutical compositions comprising the anti-CD38 antibody and a pharmaceutically acceptable carrier. The carrier may be diluent, adjuvant, excipient, or vehicle with which the anti-CD38 antibody is administered. Such vehicles may be liquids, such as water and oils, including those of petroleum, animal, vegetable, or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the molecules or antibodies of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989.

Suitable routes of administering the anti-CD38 antibody include intradermal, intramuscular, intraperitoneal, intravenous, subcutaneous, pulmonary, oral, intranasal, intravaginal, rectal, or other means known to those skilled in the art. In some embodiments, for example, the anti-CD38 antibody is administered intravenously. The anti-CD38 antibody may be administered parentally by intravenous (IV) infusion or bolus injection. IV infusion can be given over for example 15, 30, 60, 90, 120, 180, or 240 minutes, or from 1, 2, 3, 4, 5, 6, 7, 8, 9, 10, 11, or 12 hours.

The dose of anti-CD38 antibody given to a patient having CD38-positive hematological malignancy is that sufficient to alleviate or at least partially arrest the disease being treated (“therapeutically effective amount”) and includes from about 0.005 mg to about 100 mg/kg, e.g. about 0.05 mg to about 30 mg/kg or about 5 mg to about 25 mg/kg, or about 4 mg/kg, about 8 mg/kg, about 16 mg/kg, or about 24 mg/kg. Suitable doses include, for example, about 1, 2, 3, 4, 5, 6, 7, 8, 9, 10, 15, 16, 17, 18, 19, 20, 21, 22, 23, 24, 25, 30, 40, 50, 60, 70, 80, 90, or 100 mg/kg.

A fixed unit dose of the anti-CD38 antibody may also be given, for example, 50, 100, 200, 500, or 1000 mg, or the dose may be based on the patient's surface area, e.g., 500, 400, 300, 250, 200, or 100 mg/m2. Usually between 1 and 8 doses, (e.g., 1, 2, 3, 4, 5, 6, 7, or 8) may be administered to treat MM, but 9, 10, 11, 12, 13, 14, 15, 16, 17, 18, 19, 20, or more doses may be given.

The administration of the anti-CD38 antibody may be repeated after one day, two days, three days, four days, five days, six days, one week, two weeks, three weeks, one month, five weeks, six weeks, seven weeks, two months, three months, four months, five months, six months, or longer. Repeated courses of treatment are also possible, as is chronic administration. The repeated administration may be at the same dose or at a different dose. For example, the anti-CD38 antibody may be administered at 8 mg/kg or at 16 mg/kg at weekly intervals for 8 weeks, followed by administration at 8 mg/kg or at 16 mg/kg every two weeks for an additional 16 weeks, followed by administration at 8 mg/kg or at 16 mg/kg every four weeks by intravenous infusion.

The anti-CD38 antibodies may be administered by maintenance therapy, such as, e.g., once a week for a period of 6 months or more. For example, the anti-CD38 antibodies may be provided as a daily dosage in an amount of about 0.1 mg/kg to about 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19, or 20 after initiation of treatment, or any combination thereof, using single or divided doses of every 24, 12, 8, 6, 4, or 2 hours, or any combination thereof.

The anti-CD38 antibodies may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The anti-CD38 antibody and cyclophosphamide may be administered over any convenient timeframe. In some embodiments, the anti-CD38 antibody and the cyclophosphamide are administered simultaneously. In some embodiments, the anti-CD38 antibody and the cyclophosphamide are administered sequentially in any order. In some aspects, the cyclophosphamide is administered prior the anti-CD38 antibody.

In some embodiments, cyclophoshpamide is administered 300 mg/m² weekly. In some embodiments, cyclophosphamide is administered 150 mg/m² weekly. In some embociments, cyclophoshpamide is administered between 150 mg/m² and 300 mg/m² weekly. In some embodiments, cyclophsophamide is administered orally. In some embodiments, cyclophsophamide is administered for a period of 1, 2, 3, 4, 5 or 6 months. In some embodiments, cyclophosphamide si administered 40 mg/kg to 50 mg/kg intravenously divided into doses over 2-5 days. In some embodiments. In some embodiments, cyclophsophamide is administered 10-15 mg/kg orally every 7-10 days. In some embodiments, cyclophosphamide is administered 3 mg/kg-5 mg/kg orally twice weekly. In some embodiments, cyclophosphamide is administered 1 mg/kg per day to 5 mg/kg per day orally.

The anti-CD38 antibody and cyclophosphamide may be administered to a patient on the same day. Alternatively, the anti-CD38 antibody and cyclophosphamide may be administered on alternating days or alternating weeks or months, and so on. In some methods, the anti-CD38 antibody and cyclophosphamide may be administered with sufficient proximity in time that they are simultaneously present (e.g., in the serum) at detectable levels in the patient being treated. In some methods, an entire course of treatment with the anti-CD38 antibody consisting of a number of doses over a time period is followed or preceded by a course of treatment with cyclophosphamide, consisting of a number of doses. A recovery period of 1, 2, or several days or weeks may be used between administration of the anti-CD38 antibody and cyclophosphamide.

The anti-CD38 antibody in combination with cyclophosphamide may be administered together with any form of radiation therapy including external beam radiation, intensity modulated radiation therapy (IMRT) and any form of radiosurgery including Gamma Knife, Cyberknife, Linac, and interstitial radiation (e.g., implanted radioactive seeds, GliaSite balloon), and/or with surgery.

The anti-CD38 antibody may be administered as a pharmaceutical composition comprising the anti-CD38 antibody and a hyaluronidase. In some embodiments, the pharmaceutical composition comprising the anti-CD38 antibody and a hyaluronidase is administered subcutaneously. The concentration of the anti-CD38 antibody in the pharmaceutical composition may be about 20 mg/ml. The pharmaceutical composition may comprise between about 1,200 mg to about 1,800 mg of the anti-CD38 antibody. In some embodiments, the pharmaceutical composition may comprise about 1,200 mg of the anti-CD38 antibody. In some embodiments, the pharmaceutical composition may comprise about 1,600 mg of the anti-CD38 antibody. In some embodiments, the pharmaceutical composition may comprise about 1,800 mg of the anti-CD38 antibody. The pharmaceutical composition may comprise between about 30,000 U to about 45,000 U of the hyaluronidase. In some embodiments, the pharmaceutical composition may comprise about 1,200 mg of the anti-CD38 antibody and about 30,000 U of the hyaluronidase. In some embodiments, the pharmaceutical composition may comprise about 1,800 mg of the anti-CD38 antibody and about 45,000 U of the hyaluronidase. In some embodiments, the pharmaceutical composition may comprise about 1,600 mg of the anti-CD38 antibody and about 30,000 U of the hyaluronidase. In some embodiments, the pharmaceutical composition may comprise about 1,600 mg of the anti-CD38 antibody and about 45,000 U of the hyaluronidase.

The pharmaceutical composition may comprise the hyaluronidase rHuPH20 having the amino acid sequence of SEQ ID NO: 22, which is a recombinant hyaluronidase (HYLENEX® recombinant) described in Int'l Pat. Pub. No. WO2004/078140.

The administration of the pharmaceutical composition comprising the anti-CD38 antibody and the hyaluronidase may be repeated after one day, two days, three days, four days, five days, six days, one week, two weeks, three weeks, four weeks, five weeks, six weeks, seven weeks, two months, three months, four months, five months, six months, or longer. Repeated courses of treatment are also possible, as is chronic administration. The repeated administration may be at the same dose or at a different dose. For example, the pharmaceutical composition comprising the anti-CD38 antibody and the hyaluronidase may be administered once weekly for eight weeks, followed by once in two weeks for 16 weeks, followed by once in four weeks. The pharmaceutical compositions to be administered may comprise about 1,200 mg of the anti-CD38 antibody and about 30,000 U of hyaluronidase, wherein the concentration of the antibody that specifically binds CD38 in the pharmaceutical composition is about 20 mg/ml. The pharmaceutical composition comprising the anti-CD38 antibody and the hyaluronidase may be administered subcutaneously to the abdominal region.

The pharmaceutical composition comprising the anti-CD38 antibody and the hyaluronidase may be administered in a total volume of about 80 ml, 90 ml, 100 ml, 110 ml, or 120 ml. For administration, 20 mg/ml of the anti-CD38 antibody in 25 mM sodium acetate, 60 mM sodium chloride, 140 mM D-mannitol, 0.04% polysorbate 20, pH 5.5 may be mixed with rHuPH20, 1.0 mg/mL (75-150 kU/mL) in 10 mM L-Histidine, 130 mM NaCl, 10 mM L-Methionine, 0.02% Polysorbate 80, pH 6.5 prior to administration of the mixture to a subject.

The methods can further comprise administering a therapeutically effective amount of a corticosteroid. Exemplary corticosteroids include, for example, a glucocorticoid (cortisol, for example), prednisone, or dexamethasone. In some embodiments, the corticosteroid is dexamethasone. Thus, the methods can comprise administering to the subject a therapeutically effective amount of an anti-CD38 antibody, cyclophosphamide, and dexamethasone for a time sufficient to treat the CD38-positive hematological malignancy.

In some embodiments, corticosteroid is administered 80 mg weekly. In some embodiments, corticosteroid is administered 40 mg weekly. In some embodiments, corticosteroid is administered twice a week. In some embodiments, corticosteroid is administered once a week. In some embodiments, corticosteriod is administered orally. In some embodiments, corticosteroid is administered intravenously.

The methods can further comprise administering a therapeutically effective amount of a non-corticosteroid chemotherapeutic agent. Exemplary non-corticosteroid chemotherapeutic agents include glutamic acid derivatives or proteasome inhibitors. Exemplary glutamic acid derivatives include thalidomide (Thalomid®) or a thalidomide analog, e.g. CC-5013 (lenalidomide, Revlimid™), pomalidomide or CC4047 (Actimid™). In some embodiments, the glutamic acid derivative is lenalidomide. Thus, the methods can comprise administering to the subject a therapeutically effective amount of an anti-CD38 antibody, cyclophosphamide, and lenalidomide for a time sufficient to treat the CD38-positive hematological malignancy.

Exemplary proteasome inhibitors include bortezomib (Velcade®), carfilzomib, ixazomib, or vinca alkaloid, such as vincristine, or an anthracycline, such as doxorubicin. In some embodiments, the proteasome inhibitor is bortezomib. Thus, the methods can comprise administering to the subject a therapeutically effective amount of an anti-CD38 antibody, cyclophosphamide, and bortezomib for a time sufficient to treat the CD38-positive hematological malignancy.

In some embodiments, bortezomib is administered 1.5 mg/m² once a week. In some embodiments, bortezomib is administered 1.3 mg/m² once a week. In some embodiments, bortezomib is administered at about 1.3 mg/m² to about 1.5 mg/m² once a week. In some embodiments, bortezomib is administered 1.3 mg/m² twice a week. In some embodiments, bortezomib is administered by subcutaneous injection.

In some embodiments, the methods can comprise administering to the subject a therapeutically effective amount of an anti-CD38 antibody, cyclophosphamide, dexamethasone, and lenalidomide for a time sufficient to treat the CD38-positive hematological malignancy. In some embodiments, the methods can comprise administering to the subject a therapeutically effective amount of an anti-CD38 antibody, cyclophosphamide, dexamethasone, and bortezomib for a time sufficient to treat the CD38-positive hematological malignancy. In some embodiments, the methods can comprise administering to the subject a therapeutically effective amount of an anti-CD38 antibody, cyclophosphamide, dexamethasone, lenalidomide, and bortezomib for a time sufficient to treat the CD38-positive hematological malignancy.

In some embodiments, the methods comprise administering to a subject a therapeutically effective amount of an anti-CD38 antibody comprising a a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising an amino acid sequence of SEQ ID NO: 11 and cyclophosphamide for a time sufficient to treat the CD38-positive hematological malignancy, wherein
a) the anti-CD38 antibody is administered at a dose of about 16 mg/kg weekly for first two treatment cycles, at a dose of about 16 mg/kg once in two weeks for subsequent four treatment cycles and at a dose of about 16 mg/kg once in four weeks for any additional treatment cycles; and
b) cyclophosphamide is administered at a dose of about 150-300 mg/m² weekly for 4 to 8 treatment cycles;

In some embodiments,
a) the anti-CD38 antibody is administered on days 1, 8, 15 and 22 during the first two treatment cycles, on days 1 and 15 during the subsequent four treatment cycles and on day 1 during any additional treatment cycles; and
b) cyclophosphamide is administered on days 1, 8, 15 and 22. In some embodiments, the anti-CD38 antibody is administered intravenously.

In some embodiments, cyclophosphamide is administered orally.

In some embodiments, the methods comprise administering to a subject a therapeutically effective amount of an anti-CD38 antibody comprising a heavy chain CDR1 comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising an amino acid sequence of SEQ ID NO: 11, cyclophosphamide, bortezomib and dexamethasone for a time sufficient to treat the CD38-positive hematological malignancy, wherein
c) the anti-CD38 antibody is administered at a dose of about 16 mg/kg weekly for first two treatment cycles, at a dose of about 16 mg/kg once in two weeks for subsequent four treatment cycles and at a dose of about 16 mg/kg once in four weeks for any additional treatment cycles;
d) cyclophosphamide is administered at a dose of about 150-300 mg/m² weekly for 4 to 8 treatment cycles;
e) bortezomib is administered at a dose of about 1.3-1.5 mg/m² weekly for 3 weeks for 4 to 8 treatment cycles; and
f) dexamethasone is administered at a dose of about 40-80 mg weekly for 4-8 treatment cycles; wherein each treatment cycle is 28 days.

In some embodiments,
c) the anti-CD38 antibody is administered on days 1, 8, 15 and 22 during the first two treatment cycles, on days 1 and 15 during the subsequent four treatment cycles and on day 1 during any additional treatment cycles;
d) cyclophosphamide is administered on days 1, 8, 15 and 22;
e) bortezomib is administered on days 1, 8 and 15; and
g) dexamethasone is administered on days 1, 8, 15 and 22 or on days 1, 2, 8, 9, 15, 16, 22 and 23.
In some embodiments, the anti-CD38 antibody is administered 8 mg/kg on day 1 and 2 of week 1 of treatment.

In some embodiments, the anti-CD38 antiboyd is administered intravenously.

In some embodiments, cyclophosphamide is administered orally.

In some embodiments, bortezomib is administered subcutaneously.

In some embodiments, dexamethasone is administered orally.

In some embodiments, the anti-CD38 antibody comprises a VH comprising the amino acid sequence of SEQ ID NO: 4 and a VL comprising the amino acid sequence of SEQ ID NO: 5. In some embodiments, the anti-CD38 antibody comprises a heavy chain comprising the amino acid sequence of SEQ ID NO: 12 and a light chain comprising the amino acid sequence of SEQ ID NO: 13.

In some embodiments, the methods can comprise administering to the subject a therapeutically effective amount of an anti-CD38 antibody, cyclophosphamide, dexamethasone, and bortezomib for a time sufficient to treat the CD38-positive hematological malignancy.

In some embodiments, the subject has one or more of the following chromosomal abnormalities:
t(4;14)(p16;q32);
t(14;16)(q32;q23);
dell7p;
t(4;14)(p16;q32) and t(14;16)(q32;q23);
t(4;14)(p16;q32) and dell7p;
t(14;16)(q32;q23) and dell7p;
t(4;14)(p16;q32), t(14;16)(q32;q23) and dell7p;
dell3; or
t(11;14)(q13;q32).
   The chromosomal abnormalities are associated with poorer prognosis of subjects. The chromosomal abnormalities can be detected by fluorescent in situ hybridization (FISH). In most chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF, and t(11;14)(q13;q32) involves translocation of cyclin D1 CCND1. Deletion of 17p (dell7p) involves loss of the p53 gene locus, and deletion of 13 involves loss of retinoblastoma and DIS3 genes.

The subject can have naive multiple myeloma, relapsed multiple myeloma, or refractory multiple myeloma. In some embodiments, the subject has high risk refractory and/or relapsed multiple myeloma.

The methods of treatment can improve one or more outcome measurements of the subject compared to a subject receiving cyclophosphamide, bortezomib or dexamethasone, of combinations thereof. Exemplary outcome measurements comprise progression-free survival, overall response rate, very good partial response or better, complete response or better, or any combination thereof.

The anti-CD38 antibody and cyclophosphamide may be administered together with autologous hematopoietic stem cell transplant (AHSC). The anti-CD38 antibody and cyclophosphamide may be administered after autologous hematopoietic stem cell transplant (AHSC). The anti-CD38 antibody, cyclophosphamide, dexamethasone and bortezomib may be administered together with autologous hematopoietic stem cell transplant (AHSC). The anti-CD38 antibody, cyclophosphamide, dexamethasone and bortezomib may be administered after autologous hematopoietic stem cell transplant (AHSC).

Provided here are methods of enhancing daratumumab-mediated antbody-dependent cellular phagocytosis (ADCP) in a subject, comprising administering to the subject daratumumab and cyclophosphamide.

In some embodiments, cyclophosphamide is administered at a dose of about 150-300 mg/m²

In some embodiments, daratumumab is administered at a dose of about 16 mg/kg.

“Enhancing” refers to measaruable increase in daratumumab-mediated ADCP.

### EXAMPLES

The following examples are provided to further describe some of the embodiments disclosed herein. The examples are intended to illustrate, not to limit, the disclosed embodiments.

### Example 1

### Cyclophosphamide Alone or in Combination with Bortezomib or Lenalidomide Induces a Secretory Response by Multiple Myeloma Cells and Augments Daratumumab—Mediated Tumor Cell Killing by Macrophages

### Materials and Methods

Cells: The multiple myeloma cell line, MM1S, was sub-cultured in RPMI 1640 media supplemented with 10% FBS and 50 IU/ml penicillin and 50 μg/ml Streptomycin. For experiments, cells were plated at 2×10⁵ cells/ml and treated with increasing doses of cyclophosphamide ranging from 0-10 μM. Additionally, cells were treated with combinations of cyclophosphamide and lenalidomide (10 μM, 1 μM) as well as bortezomib (10 μM, 1 nM). THP-1 cells were sub-cultured in RPMI 1640 media supplemented with 10% FBS and 50 IU/ml Penicillin and 50 μg/ml streptomycin. Cells were plated at a cell density of 2×105 cells/ml for experiments.
MM1 S conditioned media: MM 1 S conditioned media was generated by incubating MM1S cells for 24 hrs with cyclophosphamide, lenalidomide and/or bortezomib at concentrations indicated above, after which the media was removed and the cells washed thoroughly. Fresh media was added and the cells were further incubated for 24 hours after collecting the conditioned media. Flow Cytometry Analysis of cell surface expression of CD38 and CD47: Following treatment of MM1S cells with the indicated doses of cyclophosphamide, cells were harvested by pipetting into single cell suspensions in FACS Buffer (PBS/2%FBS) after 24 hours. All antibodies were purchased from BD Pharmingen™ unless otherwise stated. Cells were stained with anti-CD38 (APC-conjugated) and anti-CD47 (FITC-conjugated, eBiosciences) antibodies and the level of expression of CD38 and CD47 was assessed. Sytox advanced reagents were used to assess cell death. Cells were analysed by flow cytometry. Sytox positive cells were excluded from analysis. The mean fluorescent intensity was calculated by expressing the fluorescent intensity of each sample relative to the cell size.
Assessment of FcyRI and FcyRIIa/b expression on macrophages: The THP-1 human monocytic cell line was plated at a density of 2×105 cells/ml. Cells were incubated with conditioned media from untreated (CTX 0) or cyclophosphamide treated MM1S cells at the indicated concentrations. FcγRI and FcγRlla/b expression was assessed by flow cytometry 48 hours later using anti-FcγRI (APC-conjugated) and anti- FcγRIIa/b (PE-conjugated) antibodies. The mean fluorescent intensity was calculated by expressing the fluorescent intensity of each sample relative to the cell size and subtracting the values for the isotype control/unstained samples.
Antibody dependent cellular cytotoxicity or phagocytosis assays: THP-1 macrophages were seeded into 96-well plates and allowed to adhere. MM target cells (MM1S cells) were labelled with calcein-AM (Invitrogen, C-3100) and added to the control or conditioned macrophages at an effector:target (E:T) ratio of 2:1. MM cell were pre-incubated in the presence of 1 μg/ml IgG1 anti-CD38 antibody (daratumumab) or the relevant isotype control and then added to cultures. After 18h incubation, cells were imaged by fluorescent microscopy and then detached by pipetting to single cell suspensions. Antibody-dependent phagocytosis was assessed with flow cytometry (Accuri), and quantified as follows: Phagocytosis was quantified by frequency of remaining target cells (Calcein-AM positive) with and without Ab treatment and isotype control. Elimination of target cells was calculated using the following formula: % daratumumab Specific Cell Clearance=100-100*(% calcein labelled MM cells (DARA treated)/% calcein labelled MM cells (isotype control). To confirm that the clearance was mediated by macrophage mediated phagocytosis, experimental wells containing macrophages that were pre-incubated with 1μg/ml Cytochalasin D were included. Cytochalasin D inhibits actin polymerisation and has been shown to inhibit cell phagocytosis.

### Results

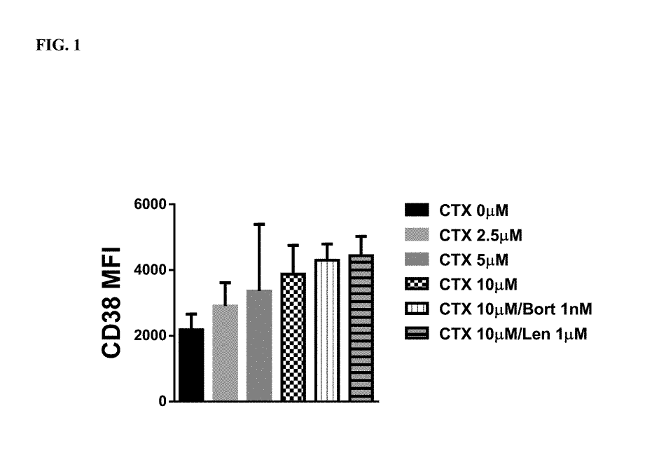

CD38 expression on multiple myeloma cells (MM1S) was induced by cyclophosphamide alone or cyclophosphamide in combination with lenalidomide or bortezomib. FIG. 1 illustrates a flow cytometry analysis of CD38 expression on MM1S cells 48 hours after treatment with cyclophosphamide alone or cyclophosphamide in combination with bortezomib or lenalidomide. The mean fluorescence intensity (MFI) of cell surface CD38 expression was calculated for MM1S cells in the absence (CTX 0) and presence of cyclophosphamide/bortezomib/lenalidomide single and combination treatments. Graphs show the mean+/− SEM for two independent experiments.

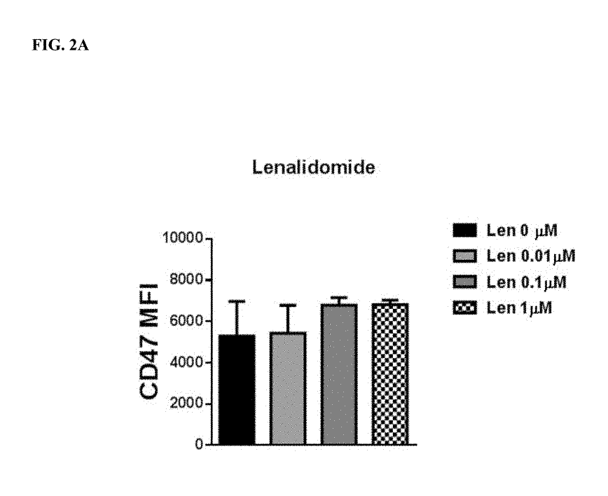

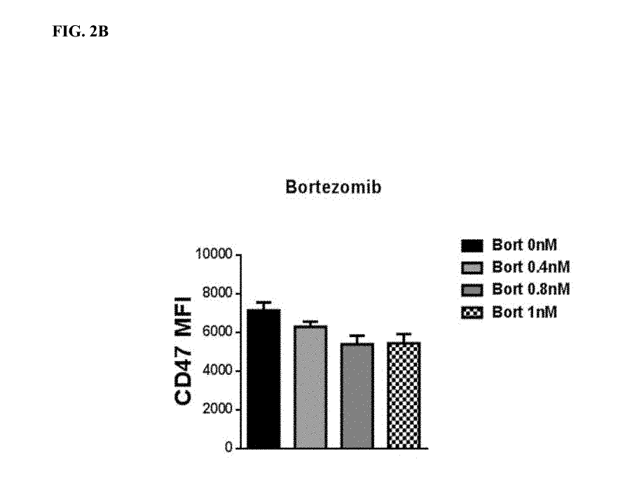

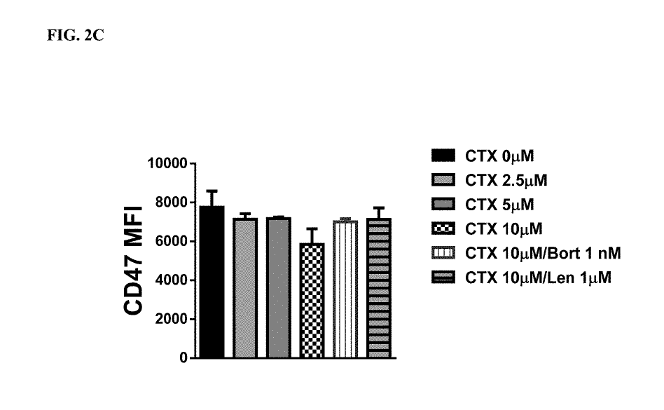

CD47 expression on multiple myeloma cells (MM1S) was reduced by cyclophosphamide. FIG. 2 illustrates flow cytometry analysis of CD47 expression on MM1S cells 24 hours after treatment with lenalidomide (FIG. 2A), bortezomib (FIG. 2B) or cyclophosphamide alone or in combination with bortezomib or lenalidomide (FIG. 2C). The mean fluorescence intensity (MFI) of cell surface CD47 expression was calculated for MM1S cells in the absence (CTX 0) and presence of cyclophosphamide/ bortezomib/lenalidomide single and combination treatments. Graphs show the mean+/− SEM for two independent experiments. As shown in FIG. 2C, CD47 expression was reduced on MM1S cells 24 hours following treatment with cyclophosphamide alone. This effect, however, was lost when cyclophosphamide treatment was combined with bortezomib or lenalidomide.

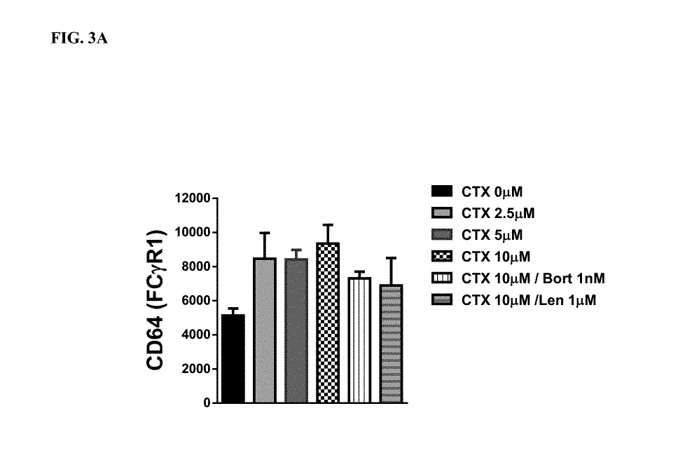

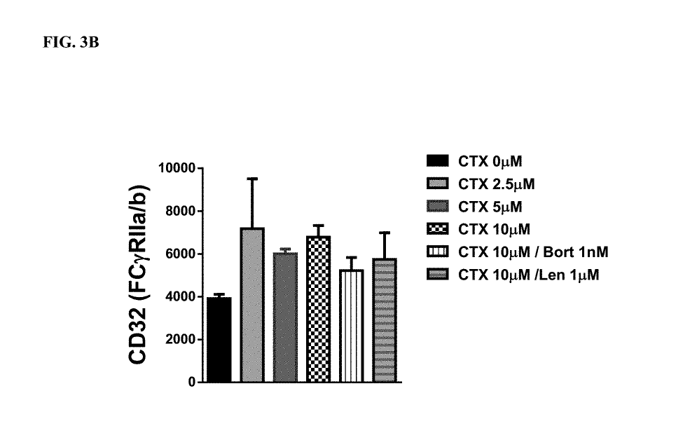

Conditioned media from cyclophosphamide-treated MM1S cells induced FcγRI and FcγRlla/b expression on macrophages. FIG. 3A illustrates flow cytometry analysis of CD64 (FcγRI) and FIG. 3B illustrates flow cytometry analysis of CD32 (FcγRlIa/b) expression on THP-1 cells 48 hours following treatment with conditioned media from untreated MM1S cells or MM1S cells treated with cyclophosphamide alone or cyclophosphamide in combination with bortezomib or lenalidomide. The mean fluorescence intensity (MFI) of cell surface CD64 and CD32 expression was calculated. Graphs show the mean+/− SEM for two independent experiments. CD64 (FcγRI) and CD32 (FcγRII) were induced on macrophages following conditioning with media from MM1S cells treated with cyclophosphamide. Although induced, this effect, was not as evident when cyclophosphamide was used in combination with bortezomib or lenalidomide.

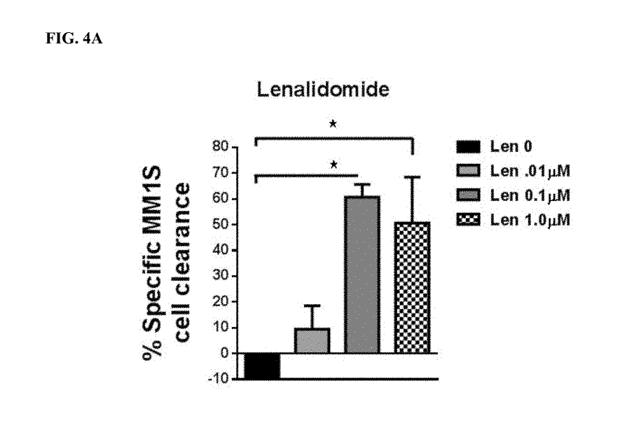

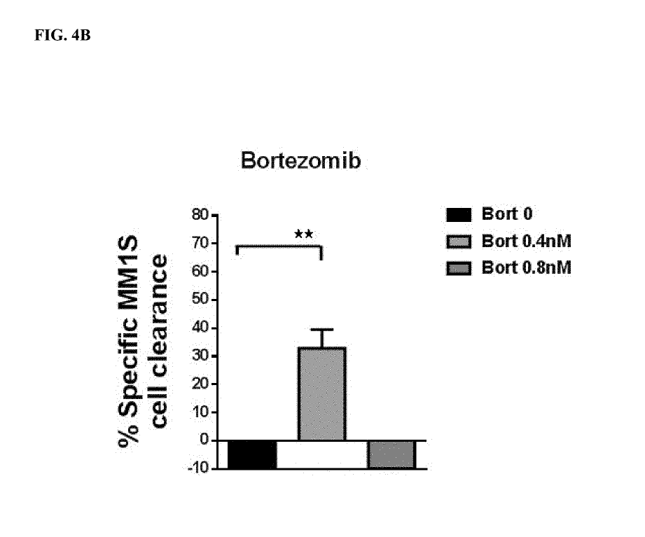

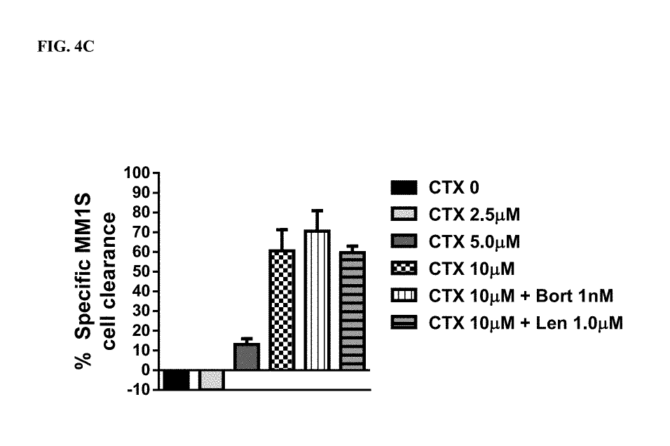

Cyclophosphamide, lenalodomide and dexamethasone augmented daratumumab-mediated tumor cell killing by macrophages. FIG. 4 illustrates the level of MM1S tumor cell clearance in co-culture of MM1S cells and THP-1 macrophages. Prior to initiation of co-cultures, THP-1 cells were cultured 48 hours in conditioned media generated from untreated MM1S tumor cells or MM1S cells treated with lenalidomide (FIG. 4A), bortezomib (FIG. 4B) or cyclophosphamide (CTX) alone or in combination with bortezomib or lenalidomide (FIG. 4C). MM1S cells were stained with calcein and incubated with daratumumab or IgG1 isotype control (1 μl) and co-culture with THP-1 cells at a 2:1 effector to target ratio for 18 hours. Cell frequency was determined using the Accuri C6 (Right). The % specific cell clearance was calculated by the following formula:

100−100*(The frequency of calcein stained MM1S cells in the presence of 1 μg/ml daratumumab/

The frequency of calcein stained MM1S cells in the presence of Isotype control). Thus, macrophage-mediated anti-myeloma activity of daratumumab was potentiated by cyclophosphamide, lenalidomide, bortezomib or cyclophosphamide in combination with lenalidomide or bortezomib.

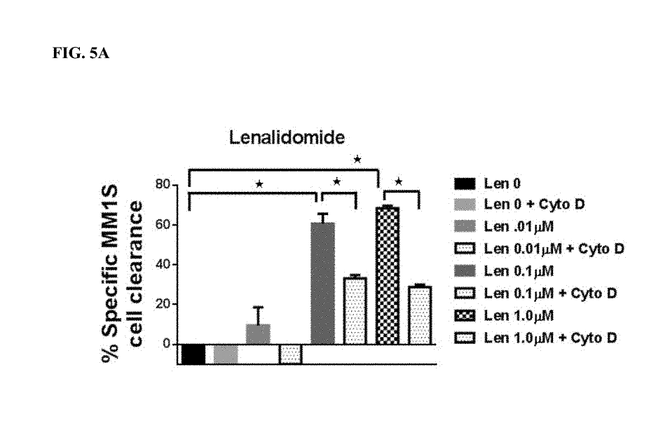

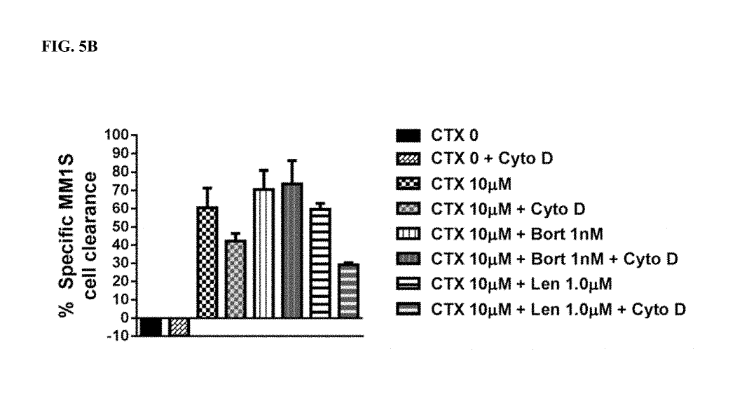

Cytochalasin D reduced cyclophosphamide, lenalodomide and dexamethasone augmented daratumumab-mediated tumor cell killing by macrophages. Co-cultures of MM1S and THP-1 cells were incubated in the presence of cytochalasin to assess if daratumumab mediated MM1S killing by ADCP. Cytochalasin D inhibits actin polymerisation and has been shown to inhibit cell phagocytosis. FIG. 5 illustrates the level of daratumumab-mediated tumor cell clearance in co-cultures of MM1S and THP-1 cells. Prior to initiation of co-cultures, THP-1 cells were cultured 48 hours in conditioned media generated from untreated MM1S tumor cells or MM1S cells treated with lenalidomide (FIG. 5A) or cyclophosphamide alone or in combination with lenalidomide or bortezomib (FIG. 5B) in the absence or presence of 1 μg/ml cytochalasin D. The % specific cell clearance was calculated by the following formula:

100−100*(The frequency of calcein stained MM1S cells in the presence of 1 μg/ml daratumumab/

The frequency of calcein stained MM1S cells in the presence of Isotype control). Potentiation of anti-myeloma activity of daratumumab with combination of cyclophosphamide or cyclophosphamide in combination with lenalidomide was therefore dependent on macrophage mediated ADCP.

### Example 2

### Daratumumab plus Cyclophosphamide, Bortezomib and Dexamethasone (Dara-CyBorD) in Previously Untreated and Relapsed Subjects with Multiple Myeloma

A Phase 2 study evaluating the combination of daratumumab and oral cyclophosphamide, bortezomib, and dexamethasone (Dara-CyBorD) in subjects with previously untreated multiple myeloma, irrespective of eligibility for high-dose chemotherapy (HDT) and autologous stem cell transplant (ASCT), or relapsed multiple myeloma following one prior line of therapy is conducted. Clinical trials identificationi number NCT02951819.

### Primary Objective

The primary objective is to evaluate the complete response+ very good partial response (CR+VGPR) rate following 4 cycles of induction therapy with daratumumab plus CyBorD (Dara-CyBorD), in previously untreated subjects, and in relapsed subjects with multiple myeloma, as defined by the International Myeloma Working Group (IMWG) criteria.

### Secondary Objectives

The secondary objectives are to evaluate, in previously untreated subjects and in relapsed subjects with multiple myeloma:
Overall response rate (CR+VGPR+partial response [PR], ORR)
   Following 4 cycles of Dara-CyBorD induction therapy
   At the end of 4 to 8 cycles of Dara-CyBorD induction therapy
   Post-transplant (in subjects who undergo high dose therapy and autologous stem cell transplantation)
   At the end of 12 cycles of daratumumab maintenance therapy
Time to VGPR or better
Time to PR or better
Progression-free survival (PFS) rate: at 1 year and 3 years
Overall survival (OS) rate: at 1 year and 3 years
Safety and tolerability of Dara-CyBorD
Safety profile of split-dose initial infusions of daratumumab administered as 8 mg/kg on Cycle 1 Day 1 (C1D1) and cycle 1 Day 2 (C1D2)

### Exploratory Objective

An exploratory objective is to evaluate the clinical efficacy of Dara-CyBorD in molecular subgroups, including: dell7p, de113, t(4;14), t(11;14), t(14;16).

### Primary Endpoint

The primary endpoint is the proportion of subjects achieving CR+VGPR response following 4 cycles of induction therapy with Dara-CyBorD, in previously untreated subjects, and in relapsed subjects with multiple myeloma, as defined by the IMWG criteria.

### Secondary Endpoints

The secondary efficacy endpoints include:
ORR—the proportion of subjects achieving CR+VGPR+PR response following 4 cycles of induction therapy with Dara-CyBorD, in previously untreated subjects, and in relapsed subjects with multiple myeloma, as defined by the IMWG criteria at the following timepoints:
   Following 4 cycles of induction therapy
   At the end of Dara-CyBorD induction therapy
   Post -transplant (in subjects who undergo high dose therapy and autologous stem cell transplantation)
   At the end of all (12 cycles) maintenance therapy
Time to VGPR or better—the duration from the date of start of induction therapy to the date of initial documentation of VGPR or better, which was confirmed by a repeated measurement as required by the IMWG criteria*
Time to PR or better—the duration from the date of start of induction therapy to the date of initial documentation of PR or better, which was confirmed by a repeated measurement as required by the IMWG criteria*
Duration of response—defined as the duration from the date of initial documentation of a response (PR or better) according to the IMWG criteria to the date of first documented evidence of progressive disease according to the IMWG criteria
PFS—defined as the duration from the date of start of induction therapy to the date of first documented evidence of progressive disease or death, whichever comes first. PFS rate at 1 year and 3 years will be estimated.
OS measured from the date of start of induction therapy to the date of the subject's death. OS rate at 1 year and 3 years will be estimated.
   The safety endpoints are:
Safety and tolerability of the combination of Dara-CyBorD as assessed by the incidence of adverse events and laboratory test abnormalities
Infusion reaction profile of split-dose infusions of daratumumab administered as 8 mg/kg on C1D1 and C1D2 by tabulating the incidence of infusion-related reactions by System-Organ Class and Preferred Term
   Predefined data censoring rules will be applied for time to event analyses.

### Exploratory Endpoint

Clinical efficacy of Dara-CyBorD in high-risk molecular subgroups including: dell7p, del13, t(4;14), t(11;14), t(14;16).

### Hypothesis

The primary hypothesis of this study is that CR+VGPR rate following 4 cycles of induction therapy with Dara-CyBorD in previously untreated subjects is higher than 60%, and in relapsed subjects with multiple myeloma is higher than 30%, as defined by the IMWG criteria.

### Study Design

This is a multicenter, single-arm, open-label, Phase 2 study evaluating the combination of daratumumab and oral cyclophosphamide, bortezomib, and dexamethasone (Dara-CyBorD) in subjects with previously untreated multiple myeloma, irrespective of eligibility for high-dose chemotherapy (HDT) and autologous stem cell transplant (ASCT), or relapsed multiple myeloma following one prior line of therapy. Relapsed multiple myeloma following one line of therapy is defined as having achieved at least a PR with first-line therapy before progression. Approximately 100 subjects will be enrolled into this study with at least 40 previously untreated multiple myeloma subjects and at least 40 subjects with relapsed multiple myeloma following one prior line of therapy.

Treatment phases consist of induction therapy with 4 to 8 cycles of Dara-CyBorD (this range allows for local standard of care and physician discretion), consolidation therapy with HDT/ASCT for eligible subjects, and maintenance therapy. Following induction and/or consolidation therapy with ASCT, subjects will receive maintenance therapy with daratumumab alone for 12 cycles (of 28 days each), or until disease progression (whichever occurs first). Maintenance therapy for subjects who have ASCT should begin approximately 90 days after ASCT. The follow-up period for each subject, after completion of maintenance therapy with daratumumab, will continue through 36 months following the start of induction therapy. Throughout the study, subjects will be monitored closely for adverse events, laboratory abnormalities, and clinical response. Disease response and progression will be based on assessments from IMWG Guidelines. Efficacy assessments include: M-protein measurements (serum and urine), immunofixation (serum and urine), serum free light chains, serum calcium corrected for albumin, serum immunoglobulins, examination of bone marrow aspirate or biopsy, and skeletal survey/documentation of extramedullary plasmacytomas. A Data Monitoring Committee will be commissioned for this study.

### Subject Population

Screening for eligible subjects will be performed within 28 days before enrollment. The inclusion and exclusion criteria for enrolling subjects in this study are described below.

### Inclusion Criteria

Each potential subject must satisfy all of the following criteria to be enrolled in the study:
Be at least 18 years of age.
Have documented multiple myeloma as defined by the IMWG 2015 criteria below:
Clonal bone marrow plasma cells ?10% or biopsy-proven bony or extramedullary plasmacytoma* and any one or more of the following CRAB (calcium level, renal dysfunction, anemia, and destructive bone lesions) features and myeloma defining events:
Myeloma defining events:
   Evidence of end organ damage that can be attributed to the underlying plasma cell proliferative disorder, specifically:
   Hypercalcemia: serum calcium>0.25 mmol/L (>1 mg/dL) higher than the upper limit of normal or>2.75 mmol/L (>11 mg/dL)
   Renal insufficiency: creatinine clearance<40 mL per min or serum creatinine>177? mol/L (>2 mg/dL)
   Anemia: hemoglobin value of>20 g/L below the lower limit of normal, or a hemoglobin value<100 g/L
   Bone lesions: one or more osteolytic lesions on skeletal radiography, CT (computed tomography), or positron emission tomography (PET)-CT**. If bone marrow has<10% clonal plasma cells, more than one bone lesion is required to distinguish from solitary plasmacytoma with minimal marrow involvement
Any one or more of the following biomarkers of malignancy:
   Clonal bone marrow plasma cell percentage*>60%
   Involved:uninvolved serum FLC ratio***>100
   >1 focal lesions on MRI (magnetic resonance imaging) studies; Each focal lesion must be 5 mm or more in size.
*Clonality should be established by showing κ/λ-light-chain restriction on flow cytometry, immunohistochemistry, or immunofluorescence. Bone marrow plasma cell percentage should preferably be estimated from a core biopsy specimen; in case of a disparity between the aspirate and core biopsy, the highest value should be used.
**If bone marrow has less than 10% clonal plasma cells, more than one bone lesion is required to distinguish from solitary plasmacytoma with minimal marrow involvement.
***These values are based on the serum Freelite assay (The Binding Site Group, Birmingham, UK). The involved FLC must be ?100 mg/L.
Have measurable disease as defined by any of the following:
   Serum M-protein level≥1.0 g/dL or urine M protein level≥200 mg/24 hours; or
   Immunoglobulins A, D, E or M multiple myeloma: serum M-protein level≥0.5 g/dL or urine M-protein level ?200 mg/24 hours; or
   Light chain multiple myeloma without measurable disease in the urine: serum Ig FLC≥10 mg/dL and abnormal serum Ig kappa/lambda FLC ratio
Have previously untreated myeloma or relapsed myeloma with one prior line of therapy including an induction regimen which may be followed by autologous stem cell transplantation and single agent maintenance therapy. For previously untreated subjects an emergency course of steroids (defined as no greater than 40 mg of dexamethasone, or equivalent per day for a maximum of 4 days) is permitted. In addition, radiation therapy is permitted prior to study entry, during screening, and during Cycles 1-2 of study treatment as needed for lytic bone disease.
Have an Eastern Cooperative Oncology Group (ECOG) performance status score of 0, 1, or 2 (refer to Attachment 2).
Before C1D1, a woman must be either:
   Not of childbearing potential: premenarchal; postmenopausal (>45 years of age with amenorrhea for at least 12 months or any age with amenorrhea for at least 6 months and a serum follicle stimulating hormone level>40 IU/L or mIU/mL]); permanently sterilized (e.g., bilateral tubal occlusion [which includes tubal ligation procedures as consistent with local regulations], hysterectomy, bilateral salpingectomy, bilateral oophorectomy); or otherwise be incapable of pregnancy,
   Of childbearing potential and practicing a highly effective method of birth control for 4 weeks before initiating study treatment that is consistent with local regulations regarding the use of birth control methods for subjects participating in clinical studies: eg, established use of oral, injected or implanted hormonal methods of contraception; placement of an intrauterine device or intrauterine system; barrier methods: condom with spermicidal foam/gel/film/cream/suppository or occlusive cap (diaphragm or cervical/vault caps) with spermicidal foam/gel/film/cream/suppository; male partner sterilization (the vasectomized partner should be the sole partner for that subject); true abstinence (when this is in line with the preferred and usual lifestyle of the subject)
      Note: If the childbearing potential changes after start of the study (eg, woman who is not heterosexually active becomes active, premenarchal woman experiences menarche) a woman must begin a highly effective method of birth control, as described above.
A woman of childbearing potential must have 2 negative serum (β human chorionic gonadotropin) or urine pregnancy tests during screening, the first one within 28 days prior to the first dose of study drug and the second within 24 hours prior to the first dose of study drug.
A man who is sexually active with a woman of childbearing potential and has not had a vasectomy must agree to use a barrier method of birth control e.g., either condom with spermicidal foam/gel/film/cream/suppository or partner with occlusive cap (diaphragm or cervical/vault caps) with spermicidal foam/gel/film/cream/suppository, and all men must also not donate sperm during the study and for 3 months after receiving the last dose of study drug.
Subjects must be willing and able to adhere to the prohibitions and restrictions specified in this protocol and referenced in the informed consent form (ICF).
Each subject (or their legally acceptable representative) must sign an ICF indicating that he or she understands the purpose of and procedures required for the study and are willing to participate in the study.

### Exclusion Criteria

Any potential subject who meets any of the following criteria will be excluded from participating in the study.
Refractory to any PI or the combination of PI and IMiD agents (such as lenalidomide), defined as failure to respond or progression within 60 days of the end of PI therapy
Diagnosed or treated for malignancy other than multiple myeloma, except:
   Malignancy treated with curative intent and with no known active disease present for ?5 years before enrollment
   Adequately treated non-melanoma skin cancer or lentigo maligna without evidence of disease
   Adequately treated carcinoma in situ (eg, cervical, breast) with no evidence of disease
Exhibiting clinical signs of or has a known history of meningeal or central nervous system involvement by multiple myeloma
Has known chronic obstructive pulmonary disease with a forced expiratory volume in 1 second (FEV1)<50% of predicted normal (Note that FEV1 testing is required for subjects suspected of having chronic obstructive pulmonary disease and subjects must be excluded if FEV1 <50% of predicted normal.)
Has known moderate or severe persistent asthma within the past 2 years, or currently has uncontrolled asthma of any classification (Note that subjects who currently have controlled intermittent asthma or controlled mild persistent asthma are allowed in the study.)
Is known to be seropositive for human immunodeficiency virus, known to have hepatitis B surface antigen positivity, or known to have a history of hepatitis C
Has any concurrent medical condition or disease (e.g., active systemic infection) that is likely to interfere with study procedures or results, or that in the opinion of the investigator would constitute a hazard for participating in this study
Has clinically significant cardiac disease, including:
   Myocardial infarction within 6 months before C1D1, or unstable or uncontrolled disease/condition related to or affecting cardiac function (e.g., unstable angina, congestive heart failure, New York Heart Association Class III-IV)
   Uncontrolled cardiac arrhythmia (NCI-CTCAE Version 4.03 Grade 2 or higher) or clinically significant electrocardiogram (ECG) abnormalities
   Screening 12-lead ECG shows a baseline QT interval as corrected by Fridericia's formula (QTcF) >470 msec
Has any of the following laboratory test results during the screening phase:
   Absolute neutrophil count≤1.0×109/L; (granulocyte colony stimulating factor use is permitted)
   Hemoglobin level≤7.5 g/dL (≤5 mmol/L); blood transfusions to maintain hemoglobin >7.5 are acceptable
   Platelet count<75×109/L for subjects in whom<50% of bone marrow nucleated cells are plasma cells; otherwise platelet count<50×109/L; no platelet transfusions in the past 7 days are allowed
   Alanine aminotransferase (ALT) level≥2.5× upper limit of normal (ULN)
   Aspartate aminotransferase (AST) level≥2.5× ULN
   Total bilirubin level≥1.5× ULN, (except for Gilbert Syndrome: direct bilirubin 2× ULN)
   Creatinine clearance≤20 mL/min estimated using Cockcroft-Gault; see Attachment 1
   Corrected serum calcium>14.0 mg/dL (>3.5 mmol/L) or free ionized calcium >6.5 mg/dL (>1.6 mmol/L)
Has known allergies, hypersensitivity, or intolerance to monoclonal antibodies or human proteins, daratumumab or its excipients, or known sensitivity to mammalian-derived products
Has plasma cell leukemia (>2.0×109/L circulating plasma cells by standard differential), Waldenstrom's macroglobulinemia, POEMS syndrome (polyneuropathy, organomegaly, endocrinopathy, monoclonal protein, and/or skin changes), or amyloid light-chain amyloidosis
Is known or suspected of not being able to comply with the study protocol (e.g., because of alcoholism, drug dependency, or psychological disorder) or the subject has any condition for which, in the opinion of the investigator, participation would not be in the best interest of the subject (e.g., compromise their well-being) or that could prevent, limit, or confound the protocol-specified assessments
Has a contraindication to the use of oral cyclophosphamide, bortezomib, or corticosteroids per local prescribing information
Has received an investigational drug (including investigational vaccines) or used an invasive investigational medical device within 4 weeks before C1D1 (except for investigational anti-myeloma agents, which cannot be taken within 2 weeks before C1D1)
Is a woman who is pregnant or breast-feeding or planning to become pregnant while enrolled in this study or within 6 months after the last dose of study drug
Has any condition for which, in the opinion of the investigator, participation would not be in the best interest of the subject (e.g., compromise the well-being) or that could prevent, limit, or confound the protocol-specified assessments
Has had major surgery within 2 weeks before C1D1, or will not have fully recovered from surgery, or has surgery planned during the time the subject is expected to participate in the study or within 2 weeks after the last dose of study drug administration. (Note: subjects with planned surgical procedures to be conducted under local anesthesia may participate. Kyphoplasty is not considered a major surgery.)

### Dosage and Administration

Approximately 100 subjects will be assigned to receive Dara-CyBorD as induction on a 28-day cycle length. All subjects will receive 4 to 8 cycles of oral cyclophosphamide 300 mg/m² on Days 1, 8, 15, and 22; subcutaneous (SC) bortezomib 1.5 mg/m² on Days 1, 8, and 15; and oral or intravenous (IV) dexamethasone 40 mg weekly. Subjects will concurrently receive daratumumab on 28-day cycles (Table 1). The initial dose of daratumumab will be given as a “split dose” of 8 mg/kg IV on C1D1 and C1D2. Starting Cycle 1 Day 8 through the completion of Cycle 2, daratumumab will be given weekly at 16 mg/kg IV. For Cycle 3 to Cycle 6, subjects will receive daratumumab 16 mg/kg IV once every 2 weeks. From Cycle 7 on, subjects will receive daratumumab 16 mg/kg IV once every 4 weeks, whether in the last induction cycles with CyBorD, or alone during the maintenance phase . Regardless of the number of induction cycles given, all eligible subjects are to receive 12 cycles of maintenance therapy with daratumumab 16 mg/kg IV monotherapy, given on 28-day cycles.

The induction regimen is the same dose, schedule and cycle length for all subjects in both study populations (previously untreated myeloma or relapsed myeloma with one prior line of therapy), although the number of induction cycles may vary from 4 to 8, based on local standard of care and physician discretion. Regardless of the number of induction cycles given, all subjects will receive maintenance therapy with daratumumab 16 mg/kg on Day 1 of a 28-day cycle for 12 cycles.

TABLE 1
Dosing Information for Dara-CyBorD by Cycle

| | Cycle 1 | | | | | | | | |
|---|---|---|---|---|---|---|---|---|---|
| | | | | Day | Day | | Cycles | Cycles | Maintenance |
| Drug (dose) | Day 1 | Day 2 | Day 8 | 15 | 22 | Cycle 2 | 3 to 6 | 7 to 8 | cycles |
| Daratumumab (8 mg/kg | X | X | X | X | X | Day | Day 1, | Day 1 | Day 1 |
| IV, Cycle 1 | | | | | | 1, 8, | 15 | | |
| Days 1 and 2; 16 mg/kg | | | | | | 15, | | | |
| IV, all | | | | | | 22 | | | |
| others) | | | | | | | | | |

| | | | | | | | |
|---|---|---|---|---|---|---|---|
| Bortezomib (1.5 mg/m²) | X | | X | X | | Up to 4 to 8 cycles, | — |
| | | | | | | Days 1, 8, 15 | |
| Cyclophosphamide | X | | X | X | X | Up to 4 to 8 cycles, | — |
| (300 mg/m²) | | | | | | Days 1, 8, 15, 22 | |
| Dexamethasone^{a} | X | X | X | X | X | Up to 4 to 8 cycles, | Day 1 |
| | | | | | | Days 1, 8, 15, 22 | |

^{a}See dexamethasone dosing table

TABLE 2
Dexamethasone Dose Schedule

| | | | Day of | Day of |
|---|---|---|---|---|
| | Day 1 | Day 1 | Infusion +1 | Infusion +2 |
| Cycle/ | (pre- | of | Day (post- | Days (post- |
| Timepoint | infusion)* | Week | infusion) | infusion) |
| Cycle 1 (Dara- | | | | |
| CyBorD) | | | | |
| Week 1 (Dara on | 20 D (IV) | — | 20 D on C1D2 | Day 3: 4 D |
| Days 1 and 2) | | | (IV) | |
| Week 2 | 20 D (IV) | — | 20 D | |
| Week 3 | 20 D (IV) | — | 20 D | |
| Week 4 | 20 D (IV) | — | 20 D | |
| Cycle 2 (Dara- | | | | |
| CyBorD) | | | | |
| Week 1 | 40 D | — | None needed⁺ | None needed⁺ |
| Week 2 | 40 D | — | None needed⁺ | None needed⁺ |
| Week 3 | 40 D | — | None needed⁺ | None needed⁺ |
| Week 4 | 40 D | — | None needed⁺ | None needed⁺ |
| Cycles 3-6 | | | | |
| (Dara- | | | | |
| CyBorD)** | | | | |
| Week 1 | 40 D | — | None needed⁺ | None needed⁺ |
| Week 2 | — | 40 D | None needed⁺ | None needed⁺ |
| Week 3 | 40 D | — | None needed⁺ | None needed⁺ |
| Week 4 | — | 40 D | None needed⁺ | None needed⁺ |
| Cycles 7-8 | | | | |
| (Dara- | | | | |
| CyBorD)** | | | | |
| Week 1 | 40 D | | None needed⁺ | None needed⁺ |
| Week 2 | — | 40 D | None needed⁺ | None needed⁺ |
| Week 3 | — | 40 D | None needed⁺ | None needed⁺ |
| Week 4 | — | 40 D | None needed⁺ | None needed⁺ |
| Daratumumab | | | | |
| Maintenance | | | | |
| (Q28 days for | | | | |
| 12 months; up | | | | |
| to 12 cycles) | | | | |
| Week 1 of each | 12 D | — | None needed⁺ | None needed⁺ |
| cycle | | | | |

IV—intravenous,
D—Dexamethasone,
Q28—every 28
*Also on Day 2 for Cycle 1 only
**Dara-CyBorD induction may be given for 4 to 8 cycles
⁺None needed unless subject experiences an infusion reaction

### Efficacy Evaluations

Disease response and progression will be assessed based on IMWG Guidelines. Daratumumab detection on serum immunofixation (IFE) has been demonstrated in subjects treated with 16 mg/kg, and may interfere with the traditional IMWG criteria of negative serum IFE for CR or stringent CR (sCR). To mitigate this interference, the sponsor has developed a reflex assay that utilizes anti-idiotype antibody to bind daratumumab and confirm its interference on IFE. For all subjects with VGPR and a negative M protein by serum M-protein quantitation by electrophoresis (SPEP), reflex IFE testing will be performed to confirm the presence of daratumumab on IFE. In addition, for subjects who have an SPEP≤0.2 g/dL and detectable IgG kappa myeloma during the study, reflex testing will also be performed to determine whether the para-protein identified on SPEP/IFE is monoclonal daratumumab or the subject's endogenous myeloma protein.

Disease evaluations must be performed on Day 1 of each cycle during the induction phase with Dara-CyBorD. Disease evaluation should be performed following ASCT, on Day 1 of the first maintenance cycle dose, for subjects who undergo ASCT. For all subjects, disease evaluation will be done on Day 1 of the maintenance phase with daratumumab and then on Day 1 of every other cycle (approximately every 56 days) or, if there are concerns for relapse, sooner.

### Safety Evaluations

Safety evaluations will include adverse event monitoring, clinical laboratory testing (hematology and serum chemistry), pregnancy testing, electrocardiogram monitoring, vital sign measurements, physical examinations, and ECOG performance status.

Since daratumumab interferes with the indirect antiglobulin test (TAT), subjects will receive a subject identification wallet card for the study that includes the blood profile (ABO, Rh, and IAT) determined before the first infusion of daratumumab along with information on the IAT interference for healthcare providers/blood banks.

### Example 3

### Phase 1b study of combination of Cyclophosphamide-Bortezomib-Dexamethasone (CyBorD) With Daratumumab (DARA) (CyBorD-Dara) in newly Diagnosed Multiple Myeloma Patients (NCT02955810)

This study is a Phase Ib open label, single arm, adaptive multicentre trial. Patients with newly diagnosed Multiple Myeloma (MM) will be treated with Cyclophosphamide-Bortezomib-Dexamethasone (CyBorD) in combination with Daratumumab (DARA).

### Study Design

The study will consist of 2 phases: The Screening Phase will extend up to 28 days prior to Cycle 1, Day 1. The Treatment Phase will be conducted in 2 parts and will extend from Cycle 1 Day 1 until treatment discontinuation. Treatment Phase, Part 1:Induction/Transplantation/Consolidation Phase. The consolidation phase of treatment will begin approximately 30-60 days after Autologous Stem Cell Transplantation (ASCT), when the patient has recovered sufficiently and engraftment is complete. Treatment Phase, Part 2: Maintenance Phase treatment until a maximum duration of 2 years, documented disease progression, death, loss to follow-up, or withdrawal of consent, whichever occurs first.

### Criteria

### Inclusion Criteria:

Each patient must sign an Informed Consent Form (ICF) indicating that he or she understands the purpose of and procedures required for the study and is willing to participate in the study.
Patient must be between 18 and<70 years of age.
Patient must have documented diagnosis of multiple myeloma requiring treatment as per IMWG updated criteria for the diagnosis of multiple myeloma and measurable disease as defined by:
   Monoclonal plasma cells in the bone marrow≥10% or presence of a biopsy proven plasmacytoma
   Measurable disease as defined by any of the following:
      IgG multiple myeloma: serum monoclonal paraprotein (M-protein) level≥1.0 g/dl or urine M-protein level≥200 mg/24 hours; or
      IgA, IgE, IgD or IgM multiple myeloma: serum M-protein level≥0.5 g/dl or urine M-protein level≥200 mg/24 hours; or
      Light chain multiple myeloma without measurable disease in the serum or the urine: serum immunoglobulin free light chain ?10 mg/dl and abnormal serum immunoglobulin kappa lambda free light chain ratio.
Newly diagnosed patient eligible for high dose therapy and autologous stem cell transplantation.
Patient must have an ECOG performance status score of 0-2.
Patient must have pre-treatment clinical laboratory values meeting the following criteria during the Screening Phase:
   Haemoglobin≥7.5 g/dl (≥5 mmol/l); prior red blood cell [RBC] transfusion or recombinant human erythropoietin use is permitted);
   absolute neutrophil count (ANC)≥1.0×109/1 (GCSF is permitted);
   AST≤2.5× upper limit of normal (ULN);
   ALT≤2.5× ULN;
   total bilirubin≤1.5× ULN (except in patients with congenital bilirubinaemia, such as Gilbert syndrome, direct bilirubin ?1.5× ULN);
   calculated creatinine clearance≥40 ml/min/1.73 m²;
   corrected serum calcium≤14 mg/dl (<3.5 mmol/l); or free ionized calcium≤6.5 mg/dl (≤1.6 mmol/l);
   platelet count≥70×109/1 for patients in whom<50% of bone marrow nucleated cells are plasma cells; otherwise platelet count>50×109/1 (transfusions are not permitted to achieve this minimum platelet count).
Patients who are women of child-bearing potential or male partners of women of childbearing potential must agree to use adequate contraception methods from signing of the informed consent form until at least 100 days after the last study drug administration.
Childbearing potential is defined as any woman who has not undergone a hysterectomy or bilateral oophorectomy; or has not been naturally post-menopausal for at least 12 consecutive months (i.e. has had menses at any time in the preceding 12 consecutive months). The investigator or a designated associate is required to advise the patient how to achieve adequate birth control. Highly effective contraception is defined in the study as methods that achieve a failure rate of less than 1% per year when used consistently and correctly. Such methods include: combined (oestrogen and progestogen containing) hormonal contraception associated with inhibition of ovulation (oral, intravaginal, transdermal) progestogen-only hormonal contraception associated with inhibition of ovulation (oral, injectable and implantable), intrauterine device (IUD), intrauterine hormone -releasing system (IUS), bilateral tubal occlusion, successfully vasectomised partner and sexual abstinence. In addition, the use of condoms by patients or their partners is required unless the woman has had a hysterectomy. Contraception will start 4 weeks before the start of therapy, will continue during therapy including dose interruptions and for 4 months after the last dose of any component of the treatment regimen.
A woman of childbearing potential must have 2 negative serum or urine pregnancy tests at

Screening, first within 10 to 14 days prior to first dose and the second within 24 hours prior to first dose.
Patient must be willing and able to adhere to the prohibitions and restrictions specified in this protocol.

### Exclusion Criteria:

Patient has received daratumumab or other anti-CD38 therapies previously.
Patient has a diagnosis of primary amyloidosis, monoclonal gammopathy of undetermined significance, or smoldering multiple myeloma. Monoclonal gammopathy of undetermined significance is defined by presence of serum M-protein<3 g/dl; absence of criteria consistent with active/symptomatic multiple myeloma as per IMWG criteria. Smoldering multiple myeloma is defined as asymptomatic multiple myeloma with absence of related organ or tissue impairment (ROTI) end organ damage.
Patient has a diagnosis of Waldenstrom's macroglobulinemia or other conditions in which IgM M-protein is present in the absence of a clonal plasma cell infiltration with lytic bone lesions.
Patient has prior or current systemic therapy or stem cell transplantation for any plasma cell dyscrasia, with the exception of an emergency use of a short course (equivalent of dexamethasone 40 mg/day for a maximum 4 days) of corticosteroids before treatment.
Patient has peripheral neuropathy or neuropathy grade 2 or higher, as defined by the National Cancer Institute Common Terminology Criteria for Adverse Events (NCI CTCAE) version 4.0.
Patient has had any prior or concurrent invasive malignancy (other than multiple myeloma) within 5 years of screening period except adequately treated basal cell or squamous cell carcinoma of the skin, carcinoma in situ of the cervix, localized prostate adenocarcinoma diagnosed ?3 years and without evidence of biochemical failure, or other cancer for which the patient has undergone potentially curative therapy and has no evidence of that disease for ?10 years.
Patient has had radiation therapy within 14 days of registration.
Patient has had plasmapheresis within 28 days of registration.
Patient is exhibiting clinical signs of meningeal involvement of multiple myeloma.
Patient has known chronic obstructive pulmonary disease (COPD) with a Forced Expiratory Volume in 1 second (FEV1)<50% of predicted normal. Note that FEV1 testing is required for patients suspected of having COPD and patients must be excluded if FEV1>50% of predicted normal.
Patient has known moderate or severe persistent asthma within the past 2 years, or currently has uncontrolled asthma of any classification. (Note that patients who currently have controlled intermittent asthma or controlled mild persistent asthma are allowed in the study).
Patient is known to be seropositive for or active human immunodeficiency virus (HIV) or known to have active hepatitis B or hepatitis C.
Patient has any concurrent medical or psychiatric condition or disease (e.g. active systemic infection, uncontrolled diabetes, acute diffuse infiltrative pulmonary disease) that is likely to interfere with the study procedures or results, or that in the opinion of the investigator, would constitute a hazard for participating in this study.
Patient has clinically significant cardiac disease, including:
   myocardial infarction within 1 year before registration, or an unstable or uncontrolled disease/condition related to or affecting cardiac function (e. g. unstable angina, congestive heart failure, New York Heart Association Class IIIIV), OR
   cardiac arrhythmia (NCI-CTCAE Version 4.0 Grade≥2) or clinically significant ECG abnormalities, and
   screening 12-lead ECG showing a baseline QT interval as corrected by Fridericia's formula (QTcF)>470 msec.
Patient has known allergies, hypersensitivity, or intolerance to boron or mannitol, corticosteroids, monoclonal antibodies or human proteins, or their excipients (refer to the DARA Investigator's Brochure), or known sensitivity to mammalian-derived products.
Patient has plasma cell leukaemia (according to WHO criterion:≥20% of cells in the peripheral blood with an absolute plasma cell count of more than 2×109/1) or POEMS syndrome (polyneuropathy, organomegaly, endocrinopathy, monoclonal protein, and skin changes).
Patient is known or suspected of not being able to comply with the study protocol (e.g. because of alcoholism, drug dependency, or psychological disorder). Patient has any condition for which, in the opinion of the investigator, participation would not be in the best interest of the patient (e.g. compromise the well-being) or that could prevent, limit, or confound the protocol-specified assessments.
Patient is a woman who is pregnant, or breast-feeding, or planning to become pregnant while participating in this study or within 4 months after the last dose of any component of the treatment regimen. Or, patient is a man who plans to father a child while included in this study or within 4 months after the last dose of any component of the treatment regimen.
Patient has had major surgery within 2 weeks before registration or will not have fully recovered from surgery, or has surgery planned during the time the patient is expected to participate in the study. Kyphoplasty is not considered major surgery.
Patient has received an investigational drug (including investigational vaccines) or used an invasive investigational medical device within 4 weeks before registration or is currently enrolled in an interventional investigational study.
Patient has contraindication to the use of any components of the treatment regimen, per the Summary of Product Characteristics.
Incidence of gastrointestinal disease that may significantly alter the absorption of oral drugs.
Patients unable or unwilling to undergo antithrombotic prophylactic treatment.

### Primary Outcome Measures:

To determine the Maximum Tolerated Dose (MTD) for cyclophosphamide and bortezomib that can be safely administered with DARA.
The rate of Complete Response (CR) post Autologous Stem Cell Transplantation (ASCT) [Efficacy assessed by the rate of Complete Response (CR) post Autologous Stem Cell Transplantation (ASCT)]

### Secondary Outcome Measures:

Safety and Tolerability as assessed by adverse events Safety and Tolerability will be assessed by standard clinical and laboratory tests. Adverse event grades will be determined by the NCI CTCAE v4.03
Complete Response Rate at the end of induction, ASCT, consolidation and maintenance
Best Overall Response
Minimal Residual Disease (MRD) negative rate at the end of induction, ASCT, consolidation and maintenance
Progression Free Survival (PFS) at the end of maintenance phase
Overall Survival (OS) at the end of maintenance phase
Clinical Benefit Rate (CBR)

### Dosing:

### Daratumumab

Daratumumab [DARA] (16 mg/kg) will be administered by intravenous (IV) infusion once every week for 8 weeks (CyBorD Induction Cycle 1-2), then once every 2 weeks for 8 weeks (CyBorD Induction Cycle 3-4), and following transplantation once every 2 weeks for 8 weeks (CyBorD Consolidation Cycle 5-6). For the maintenance phase, patients with high risk disease will receive bortezomib as SC injection once every two weeks plus DARA (16 mg/kg IV) once every four weeks. The dose of bortezomib used for maintenance should be the maximum tolerated dose at the end of consolidation treatment but no more than 1.3 mg/m². Patients with standard risk disease will receive DARA (16 mg/kg IV) once every four weeks. Patients will receive maintenance treatment for a maximum duration of 2 years, documented disease progression, death, loss to follow-up, or withdrawal of consent, whichever occurs first.

### CyBorD (Cyclophosphamide, Bortezomib and Dexamethasone):

Cyclophosphamide will be administered 150-300 mg/m² orally on days 1, 8, 15, 22 for four 28-day induction cycles (Cycles 1-4), and two consolidation cycles (Cycles 5-6).

Bortezomib will be administered at either a dose of 1.3 mg/m²(dose level 1 and 2) or 1.5 mg/m² (dose level 3) as a SC injection once per week (Days 1, 8, 15 and 22) for four 28-day induction cycles (Cycles 1 to 4), and two consolidation cycles (Cycles 5 and 6).

Dexamethasone: Induction phase: Cycles 1-2: Dexamethasone will be administered orally on days 1-2, 8-9, 15-16, 22-23 at 40mg per day (i.e. total of 320mg per cycle). Cycles 3-4: Dexamethasone will be administered orally on days 1-2 at 40mg per day and subsequently 20 mg per day on days 8-9, 15-16, 22-23 (i.e. total of 200mg per cycle). Consolidation phase: Cycles 5-6: Dexamethasone will be administered orally on days 1-2, 8-9, 15-16, 22-23 at 20 mg per day (i.e. total of 160 mg per cycle). Maintenance phase: In the maintenance phase, dexamethasone at 20 mg or substitutions will be administered either intravenously or orally as pre-medication on DARA infusion days, 1 hour or less prior to the DARA infusion. Dexamethasone tablets are to be taken with or immediately after a meal or snack, preferably in the morning.

### Patient Sample Collection and Analyses

Ethics: Pre-screened newly diagnosed MM patients were enrolled into the sub-clinical study by informed consent according to predetermined protocols and standard operating procedures for sample draw and included extraction of relevant details from medical records. Follow up samples were also attained after 24 hours Cyclophosphamide (150-300 mg/m2) treatment as outlined in Cyclophosphamide-Bortezomib-Dexamethasone with Daratumumab (Dara-CyBorD) clinical trial (NCT02955810) protocol. The sub-study protocol, including the information leaflets and consent forms, the potential risks and measures to prevent incidences of risk was reviewed and approved by the Galway University Hospital Ethics Committee.

Samples Isolation: Isolation of mononuclear cells (MC) from PB and BM aspirates were performed by layering 3mL EDTA-anti-coagulated sample over 3mL endotoxin-free Ficoll Plaque density-gradient medium (GE Healthcare, Little Chalfont, United Kingdom) in 15-mL Falcon tubes (Sarstedt, North Rhine-Westphalia, Niimbrecht, Germany), and centrifuged at 420 relative centrifugal force (RCF) for 22 minutes at 4° C. without braking. The “buffy coat” layer was isolated and washed twice with Fluorescence-activated cell sorting (FACS buffer) [2% foetal calf serum (Lonza, Basel, Switzerland), PBS and 0.05% NaN3 (Sigma-Aldrich, St. Louis, Mo., USA)]. The MCs were pelleted by centrifugation at 300 RCF for 10 minutes at 4° C. with full acceleration and brake. Cells were counted using a haemocytometer and 5×105 cells per sample were stained for phenotypic characterisation by flow cytometry

Characterisation Studies: Plasma cells: Cell surface expression of CD38, CD47, CD319 (SLAMF7), B-cell maturation antigen (BCMA), MIC A/B, Programmed death-ligand (PDL) 1, PDL2, CD54, CD11a and CD48 were analysed on CD45-CD19-CD138+ Plasma cells by flow cytometry. Macrophages: Cell surface expression of CD32, CD64, SIRPla, PD-1 and CD163 were analysed on CD45+CD33+CX3CR1+−CD56-CD14+ macrophages by flow cytometry. Cells were stained with flourochrome-conjugated anti-human monoclonal antibodies for 20 minutes at 4° C. All antibodies were purchased from BD Biosciences unless otherwise stated. Cells were washed with FACS buffer and the level of surface expression was assessed for each cell population by flow cytometry. The mean fluorescent intensity (MFI) was calculated by expressing the fluorescent intensity of each sample relative to the cell size. Fluorescence compensation was set using single-stained controls, and matching median compensation algorithms were applied. Fluorescence minus one controls were used to set analysis gates controls and data were analyzed using Diva v8.0.1 acquisition software (BD Biosciences) or FlowJo® 7.6.5 software (TreeStar Inc., Olten, Switzerland).

Antibody Dependant cellular phagocytosis (ADCP): To determine the number of macrophages per sample, 50 μl of 5×10⁶ cells/mL concentration of MC isolated from PB and BM were diluted using 150 μl FACS buffer. Based on forward and side scatter profiles, the total number of macrophages were determined by timed acquisition events per unit volume using an Accuri™ C6. PB and BM isolated macrophages were seeded into 96-well plates and allowed to adhere. MM target cells (MM1S cells) were labelled with CFSE (Thermofisher Scientific, Mass. USA) and pre-incubated in the presence of 1 μg/ml IgG1 anti-CD38 antibody (daratumumab) or the relevant isotype control. MM cells were then added to cultures at an effector:target (E:T) ratio of 2:1. After 12 h incubation, cells were imaged by fluorescent microscopy and then detached by pipetting to single cell suspensions. Cells were washed, incubated with mouse anti-human CD45-APC (BD Biosciences, San Jose, CA) and mouse anti-human CD14-PerCP-Cy5.5 (Miltenyi Biotech, Cologne, Germany) for 15 mins, then washed and re-suspended in 200 μl FACS buffer for sample acquisition. Macrophage and MM1S cells were gated using CFSE+ and CD45+CD14+ cells gating. Cell frequency was determined using the BD Canto IITM flow cytometer. Antibody-dependent phagocytosis was assessed and quanti?ed as follows: Phagocytosis was quanti?ed by frequency of remaining target cells (CFSE positive) with and without Ab treatment or isotype control. To confirm that the clearance was macrophage mediated phagocytosis, experimental wells containing macrophages that were pre-incubated with 1 μg/ml Cytochalasin D were included. Cytochalasin D inhibits actin polymerisation and has been shown to inhibit cell phagocytosis. ADCP mediated clearance was calculated using the following formula: 100-100*(Proportion of live Dara-treated MM1S cells in untreated macrophage co-culture/ Proportion of live Dara-treated MM1S cells in CytoD conditioned macrophage co-culture).

### RESULTS

Patients enrolled in the clinical trial were treated with (1) cyclophosphamide 150 mg/m² (first 3 patients only) then 300 mg/m² weekly (remaining patients), (2) bortezomib 1.3 mg/m² (first 6 patient only) or 1.5 mg/m² (remaining patients) and weekly dexamethasone, as part of a 28 day cycle. Daratumumab was administered as described above. For Cycle 1 day 1, bortezomib was administered 24 hours after cyclophosphamide administration.

Plamsa cell and macrophage phenotypes in patient peripheral blood (PB) and bone marrow (BM) at baseline and after 24 hour treatment (patient had received cyclophosphamide, daratumumab and dexamethasone) were analyzed.

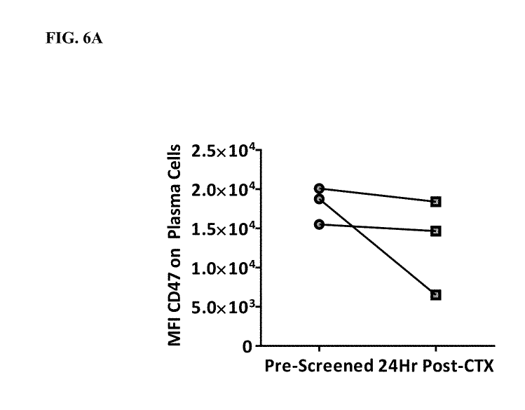

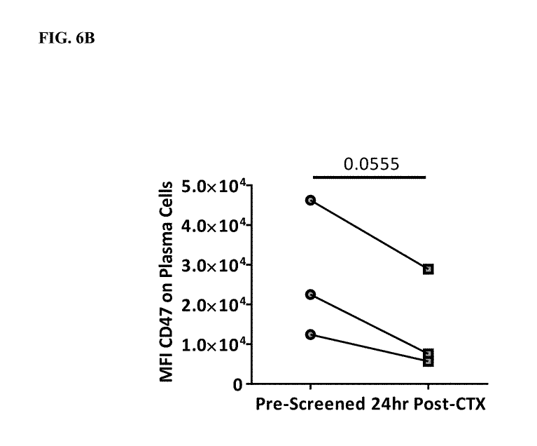

CD47 surface expression was evaluated on CD45−CD19−CD138+ plasma cells and found to be decreased on the plasma cells both in PB (FIG. 6A) and BM (FIG. 6B) after 24 hour treatment when compared to baseline. Patient CD45-CD19-CD138+ plasma cells were identified in PB and BM by multi-color flow cytometry. FIG. 6A and FIG. 6B shows the mean fluorescence intensity (MFI) of cell surface CD47 expression from 3 subjects in 3 independent experiments. These data corroborate the in vitro studies (FIG. 2C) indicating that cyclophosphamide reduced CD47 expression on MM cells.

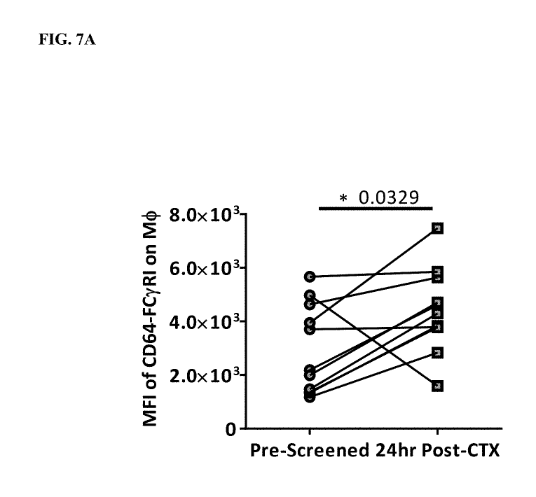

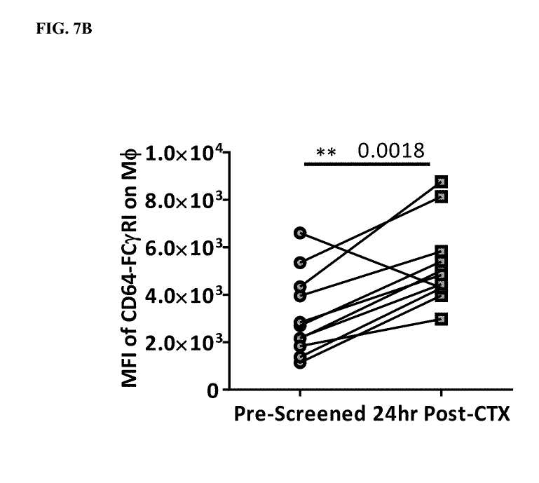

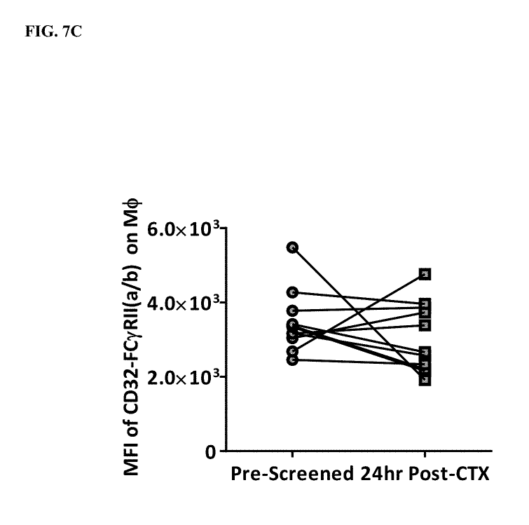

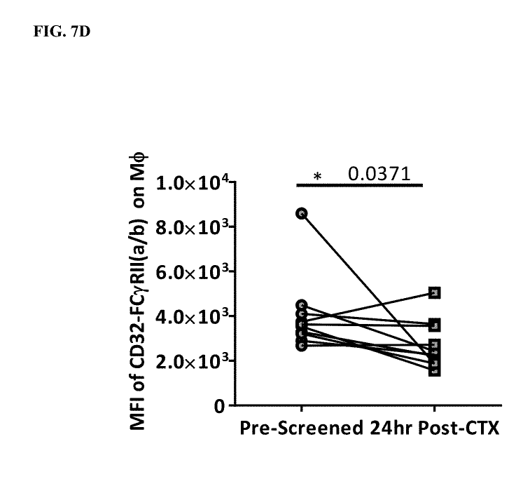

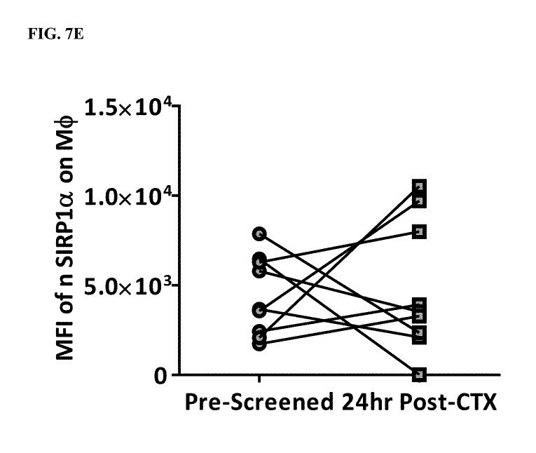

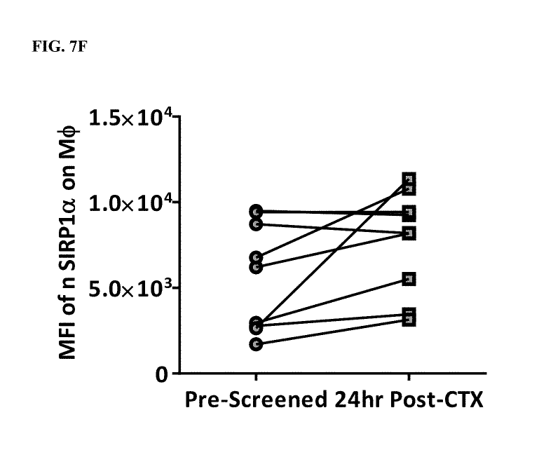

CD64 (FcyRI) surface expression was increased in macrophage precursors (CD45+CD56−CD33+CX3CR1+CD14+ cells) identified in patient PB (FIG. 7A) and BM (FIG. 7B) by 24-hour treatment with cyclophosphamide, dexamethasone and daratumumab when compared to the baseline (p=0.0329 peeripehral blood macrophages; p=0.0018 bone marrow macrophages). CD45+CD56−CD33+CX3CR1+CD14+ macrophages were identified in PB and BM by multi-colour flow cytometry. These data corroborate the in vitro findings that treatment with cyclophosphamide increased CD64 expression on macrophages.

CD32 (FcγRIIA) surface expression was not changed in the macrophage precursors (CD45+CD56−CD33+CX3CR1+CD14+ cells) in PB 24 hours post-treatment with cyclophosphamide, dexamethasone and daratumumab (FIG. 7C) however the expression was decreased in macrophage precursors after treatment vs. baseline in BM (p=0.0371 baseline vs. post-treatment) (FIG. 7D). Additionally, no change was detected in the surface expression of SIRP la on macrophages in either PB (FIG. 7E) or BM (FIG. 7F) following treatment

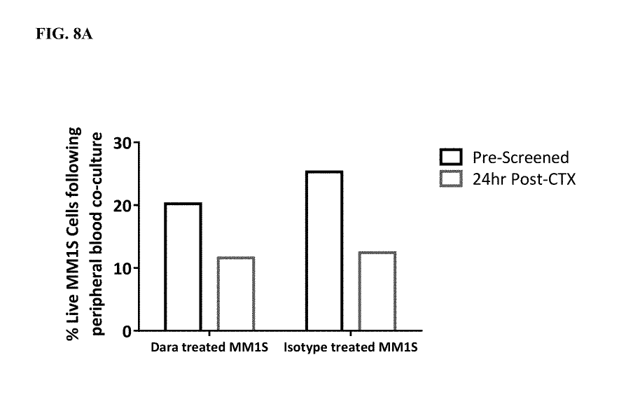

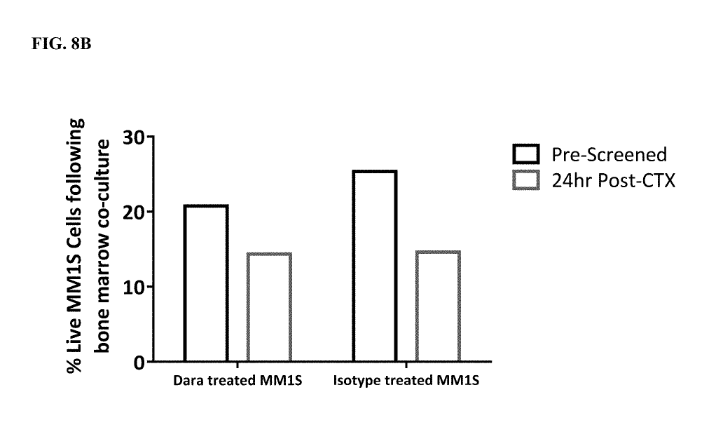

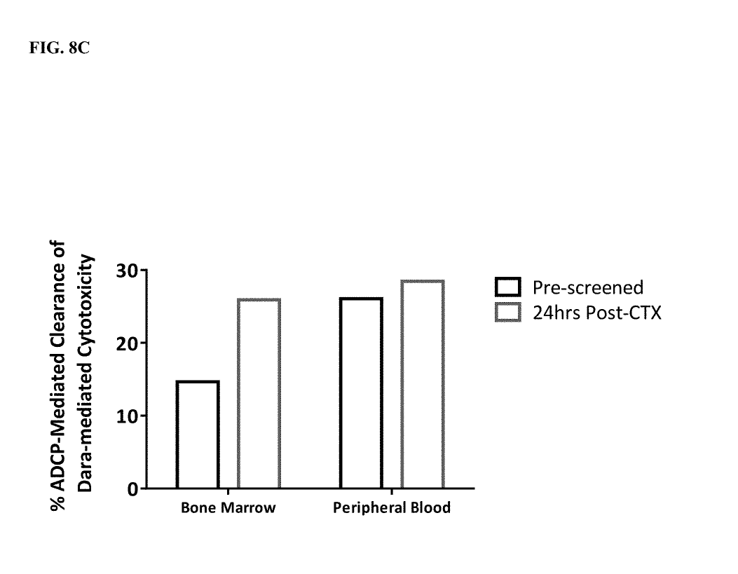

Cyclophosphamide also augmented tumour cytotoxicity by macrophages in co-culture of PB or BM-derived macrophages and multiple myeloma cells. FIG. 8A illustrates that the proportion of live Dara treated-MM1S were reduced following co-culture with PB macrophages isolated from a patient 24 hours after treatment with combinations of cyclophosphamide, dexamethasone and daratumumab compared to macrophages isolated prior to receiving treatment. FIG. 8B illustrates that the proportion of live Dara treated-MM1S were reduced following co-culture with BM macrophages isolated from a patient 24 hours after treatment with combinations of cyclophosphamide, dexamethasone and daratumumab compared to macrophages isolated prior to receiving treatment.

Co-cultures of MM1S and CD45+CD14+ macrophages from PB and BM were incubated in the presence of Cytochalasin D to assess if Daratumumab mediated MM1S killing occurs via ADCP. ADCP accounted for>15% of tumour cell clearance and was enhanced to>25% 24 hours after treatment with cyclophosphamide in BM (FIG. 8C). A similar trend was observed to a lesser degree when cultured with macrophages from PB. FIG. 8C shows 1 independent patients sampled prior to and following treatment. These data support previous findings that cyclophosphamide augments macrophage mediated ADCP to enhance anti-tumour cytotoxicity.

To note, Daratumumab (16 mg/kg) infusion was administered to the patient 1 hour after cyclophosphamide. The minimal difference between Dara and Isotype within the ADCP assay (FIG. 8C) may be due to in vivo saturation of the CD38 receptor by Dara treatment. Even so, Cycle 1 day 2 macrophages showed advanced ability to clear MM1S cells when compared to macrophages isolated from the same patient prior to receiving the drug treatment.

Together, these data highlight that a detectable increase in CD64 surface expression by macrophages isolated from adults following cyclophosphamide treatment is coupled with an increased capability for ADCP-mediated cytotoxicity of MM cells. No changes in macrophage expression was observed in the “don't eat me receptor” inhibitory receptor SIRP1 however a decreased in the antigen for this receptor, CD47 was decreased on plasma cells in these patients. This data provides a strong rationale that cyclophosphamide can potentiate the anti-tumour effect of Dara by augmentation of macrophage-mediated ADCP.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

| SEQ ID NO: | Sequence |
|---|---|
| Human CD38 aa | MANCEFSPVSGDKPCCRLSRRAQLCLGVSILVLILVVVLAVVVP |
| SEQ ID NO: 1 | RWRQQWSGPGTTKRFPETVLARCVKYTEIHPEMRHVDCQSVW |
| | DAFKGAFISKHPCNITEEDYQPLMKLGTQTVPCNKILLWSRIKD |
| | LAHQFTQVQRDMFTLEDTLLGYLADDLTWCGEFNTSKINYQSC |
| | PDWRKDCSNNPVSVFWKTVSRRFAEAACDVVHVMLNGSRSKI |
| | FDKNSTFGSVEVHNLQPEKVQTLEAWVIHGGREDSRDLCQDPTI |
| | KELESIISKRNIQFSCKNIYRPDKFLQCVKNPEDSSCTSEI |
| Human CD38 region | SKRNIQFSCKNIYR |
| 1 aa | |
| SEQ ID NO: 2 | |
| Human CD38 region | EKVQTLEAWVIHGG |
| 2 aa | |
| SEQ ID NO: 3 | |
| Daratumumab VH | EVQLLESGGGLVQPGGSLRLSCAVSGFTFNSFAMSWVRQAPGK |
| SEQ ID NO: 4 | GLEWVSAISGSGGGTYYADSVKGRFTISRDNSKNTLYLQMNSL |
| | RAEDTAVYFCAKDKILWFGEPVFDYWGQGTLVTVSS |
| Daratumumab VL | EIVLTQSPATLSLSPGERATLSCRASQSVSSYLAWYQQKPGQAP |
| SEQ ID NO: 5 | RLLIYDASNRATGIPARFSGSGSGTDFTLTISSLEPEDFAVYYCQ |
| | QRSNWPPTFGQGTKVEIK |
| Daratumumab | SFAMS |
| HCDR1 | |
| SEQ ID NO: 6 | |
| Daratumumab | AISGSGGGTYYADSVKG |
| HCDR2 | |
| SEQ ID NO: 7 | |
| Daratumumab | DKILWFGEPVFDY |
| HCDR3 | |
| SEQ ID NO: 8 | |
| Daratumumab | RASQSVSSYLA |
| LCDR1 | |
| SEQ ID NO: 9 | |
| Daratumumab | DASNRAT |
| LCDR2 | |
| SEQ ID NO: 10 | |
| Daratumumab | QQRSNWPPTF |
| LCDR3 | |
| SEQ ID NO: 11 | |
| Daratumumab HC | EVQLLESGGGLVQPGGSLRLSCAVSGFTFNSFAMSWVRQAPGK |
| SEQ ID NO: 12 | GLEWVSAISGSGGGTYYADSVKGRFTISRDNSKNTLYLQMNSL |
| | RAEDTAVYFCAKDKILWFGEPVFDYWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVD |
| | KRVEPKSCDKTHTCPPCPAPELLGGPSVFLFPPKPKDTLMISRTP |
| | EVTCVVVDVSHEDPEVKFNWYVDGVEVHNAKTKPREEQYNST |
| | YRVVSVLTVLHQDWLNGKEYKCKVSNKALPAPIEKTISKAKGQ |
| | PREPQVYTLPPSREEMTKNQVSLTCLVKGFYPSDIAVEWESNGQ |
| | PENNYKTTPPVLDSDGSFFLYSKLTVDKSRWQQGNVFSCSVMH |
| | EALHNHYTQKSLSLSPGK |
| Daratumumab LC | EIVLTQSPATLSLSPGERATLSCRASQSVSSYLAWYQQKPGQAP |
| SEQ ID NO: 13 | RLLIYDASNRATGIPARFSGSGSGTDFTLTISSLEPEDFAVYYCQ |
| | QRSNWPPTFGQGTKVEIKRTVAAPSVFIFPPSDEQLKSGTASVVC |
| | LLNNFYPREAKVQWKVDNALQSGNSQESVTEQDSKDSTYSLSS |
| | TLTLSKADYEKHKVYACEVTHQGLSSPVTKSFNRGEC |
| mAb003 VH | QVQLVQSGAEVKKPGSSVKVSCKASGGTFSSYAFSWVRQAPGQ |
| SEQ ID NO: 14 | GLEWMGRVIPFLGIANSAQKFQGRVTITADKSTSTAYMDLSSLR |
| | SEDTAVYYCARDDIAALGPFDYWGQGTLVTVSSAS |
| mAb003 VL | DIQMTQSPSSLSASVGDRVTITCRASQGISSWLAWYQQKPEKAP |
| SEQ ID NO: 15 | KSLIYAASSLQSGVPSRFSGSGSGTDFTLTISSLQPEDFATYYCQQ |
| | YNSYPRTFGQGTKVEIK |
| mAb024 VH | EVQLVQSGAEVKKPGESLKISCKGSGYSFSNYWIGWVRQMPGK |
| SEQ ID NO: 16 | GLEWMGIIYPHDSDARYSPSFQGQVTFSADKSISTAYLQWSSLK |
| | ASDTAMYYCARHVGWGSRYWYFDLWGRGTLVTVSS |
| mAb024 VL | EIVLTQSPATLSLSPGERATLSCRASQSVSSYLAWYQQKPGQAP |
| SEQ ID NO: 17 | GLLIYDASNRASGIPARFSGSGSGTDFTLTISSLEPEDFAVYYCQ |
| | QRSNWPLTFGGGTKVEIK |
| MOR-202 VH | QVQLVESGGGLVQPGGSLRLSCAASGFTFSSYYMNWVRQAPG |
| SEQ ID NO: 18 | KGLEWVSGISGDPSNTYYADSVKGRFTISRDNSKNTLYLQMNS |
| | LRAEDTAVYYCARDLPLVYTGFAYWGQGTLVTVSS |
| MOR-202 VL | DIELTQPPSVSVAPGQTARISCSGDNLRHYYVYWYQQKPGQAP |
| SEQ ID NO: 19 | VLVIYGDSKRPSGIPERFSGSNSGNTATLTISGTQAEDEADYYCQ |
| | TYTGGASLVFGGGTKLTVLGQ |
| Isatuximab VH | QVQLVQSGAEVAKPGTSVKLSCKASGYTFTDYWMQWVKQRP |
| SEQ ID NO: 20 | GQGLEWIGTIYPGDGDTGYAQKFQGKATLTADKSSKTVYMHLS |
| | SLASEDSAVYYCARGDYYGSNSLDYWGQGTSVTVSS |
| Isatuximab VL | DIVMTQSHLSMSTSLGDPVSITCKASQDVSTVVAWYQQKPGQS |
| SEQ ID NO: 21 | PRRLIYSASYRYIGVPDRFTGSGAGTDFTFTISSVQAEDLAVYYC |
| | QQHYSPPYTFGGGTKLEIK |
| SEQ ID NO: 22 | MGVLKFKHIFFRSFVKSSGVSQIVFTFLLIPCCLTLNFRAPPVIPN |
| hyaluronidase | VPFLWAWNAPSEFCLGKFDEPLDMSLFSFIGSPRINATGQGVTIF |
| rHuPH20 | YVDRLGYYPYIDSITGVTVNGGIPQKISLQDHLDKAKKDITFYM |
| | PVDNLGMAVIDWEEWRPTWARNWKPKDVYKNRSIELVQQQN |
| | VQLSLTEATEKAKQEFEKAGKDFLVETIKLGKLLRPNHLWGYY |
| | LFPDCYNHHYKKPGYNGSCFNVEIKRNDDLSWLWNESTALYPS |
| | IYLNTQQSPVAATLYVRNRVREAIRVSKIPDAKSPLPVFAYTRIV |
| | FTDQVLKFLSQDELVYTFGETVALGASGIVIWGTLSIMRSMKSC |
| | LLLDNYMETILNPYIINVTLAAKMCSQVLCQEQGVCIRKNWNSS |
| | DYLHLNPDNFAIQLEKGGKFTVRGKPTLEDLEQFSEKFYCSCYS |
| | TLSCKEKADVKDTDAVDVCIADGVCIDAFLKPPMETEEPQIFYN |
| | ASPSTLSATMFIVSILFLIISSVASL |

What is claimed:

1) A method of treating a subject having a CD38-positive hematological malignancy, comprising administering to the subject a therapeutically effective amount of an anti-CD38 antibody and cyclophosphamide for a time sufficient to treat the CD38-positive hematological malignancy.

2) The method of claim 1, wherein the CD38-positive hematological malignancy is multiple myeloma, acute lymphoblastic leukemia, diffuse large B-cell lymphoma, Burkitt's lymphoma, follicular lymphoma, mantle-cell lymphoma, acute myeloid leukemia, chronic lymphocytic leukemia, or combinations thereof.

3) The method of claim 2, wherein the CD38-positive hematological malignancy is multiple myeloma.

4) The method of claim 2, wherein the anti-CD38 antibody comprises a heavy chain complementarity determining region 1 (CDR1) comprising the amino acid sequence of SEQ ID NO: 6, a heavy chain CDR2 comprising the amino acid sequence of SEQ ID NO: 7, a heavy chain CDR3 comprising the amino acid sequence of SEQ ID NO: 8, a light chain CDR1 comprising the amino acid sequence of SEQ ID NO: 9, a light chain CDR2 comprising the amino acid sequence of SEQ ID NO: 10, and a light chain CDR3 comprising the amino acid sequence of SEQ ID NO: 11.

5) The method of claim 4, wherein the anti-CD38 antibody comprises a heavy chain variable region comprising the amino acid sequence of SEQ ID NO: 4 and a light chain variable region comprising the amino acid sequence of SEQ ID NO: 5.

6) The method of claim 5, wherein the anti-CD38 antibody comprises a heavy chain comprising the amino acid sequence of SEQ ID NO: 12 and a light chain comprising the amino acid sequence of SEQ ID NO: 13.

7) The method of claim 2, wherein the anti-CD38 antibody comprises a HCDR1, a HCDR2, a HCDR3, a LCDR1, a LCDR2 and a LCDR3 of:
a) a VH comprising the amino acid sequence of SEQ ID NO: 14 and a VL comprising the amino acid sequence of SEQ ID NO: 15;
b) a VH comprising the amino acid sequence of SEQ ID NO: 16 and a VL comprising the amino acid sequence of SEQ ID NO: 17;
c) a VH comprising the amino acid sequence of SEQ ID NO: 18 and a VL comprising the amino acid sequence of SEQ ID NO: 19; or
d) a VH comprising the amino acid sequence of SEQ ID NO: 20 and a VL comprising the amino acid sequence of SEQ ID NO: 21.

8) The method of claim 7, wherein the anti-CD38 antibody comprises:
a) the VH comprising the amino acid sequence of SEQ ID NO: 14 and the VL comprising the amino acid sequence of SEQ ID NO: 15;
b) the VH comprising the amino acid sequence of SEQ ID NO: 16 and the VL comprising the amino acid sequence of SEQ ID NO: 17;
c) the VH comprising the amino acid sequence of SEQ ID NO: 18 and the VL comprising the amino acid sequence of SEQ ID NO: 19; or
d) the VH comprising the amino acid sequence of SEQ ID NO: 20 and the VL comprising the amino acid sequence of SEQ ID NO: 21.

9) The method of claim 4, wherein the anti-CD38 antibody is an IgG1, IgG2, IgG3 or IgG4 isotype.

10) The method of claim 9, wherein the anti-CD38 antibody is the G1 isotype.

11) The method of claim 4, wherein cyclophosphamide enhances the anti-CD38 antibody-mediated antibody-dependent cell phagocytosis.

12) The method of claim 4, wherein cyclophosphamide is administered at a dose of about 150-300 mg/m² weekly for 4 to 8 treatment cycles, wherein each treatment cycle is 28 days.

13) The method of claim 12, wherein the anti-CD38 antibody is administered at a dose of about 16 mg/kg weekly for first two treatment cycles, at a dose of about 16 mg/kg once in two weeks for subsequent four treatment cycles and at a dose of about 16 mg/kg once in four weeks for any additional treatment cycles.

14) The method of claim 4, wherein the anti-CD38 antibody is administered subcutaneously in a pharmaceutical composition comprising the anti-CD38 antibody and a hyaluronidase.

15) The method of claim 14, wherein the hyaluronidase is rHuPH20 of SEQ ID NO: 22.

16) The method of claim 13, further comprising administering a therapeutically effective amount of a corticosteroid.

17) The method of claim 16, wherein the corticosteroid is dexamethasone.

18) The method of claim 17, wherein dexamethasone is administered at a dose of about 40-80 mg weekly for 4-8 treatment cycles.

19) The method of claim 18, further comprising administering a therapeutically effective amount of a non-corticosteroid chemotherapeutic agent.

20) The method of claim 19, wherein the non-corticosteroid chemotherapeutic agent comprises a glutamic acid derivative or a proteasome inhibitor.

21) The method of claim 20, wherein the glutamic acid derivative is lenalidomide.

22) The method of claim 21, wherein the proteasome inhibitor is bortezomib.

23) The method of claim 22, wherein bortezomib is administered at a dose of about 1.3-1.5 mg/m² weekly for 3 weeks for 4 to 8 treatment cycles.

24) The method of claim 23, wherein
a) the anti-CD38 antibody is administered on days 1, 8, 15 and 22 during the first two treatment cycles, on days 1 and 15 during the subsequent four treatment cycles and on day 1 during the any additional treatment cycles;
b) cyclophosphamide is administered on days 1, 8, 15 and 22 during each 4-8 treatment cycles;
c) bortezomib is administered on days 1, 8 and 15 during each 4-8 treatment cycles; and
d) dexamethasone is administered on days 1, 8, 15 and 22 or on days 1, 2, 8, 9, 15, 16, 22 and 23 during each 4-8 treatment cycles.

25) The method of claim 4, wherein the subject has one or more chromosomal abnormalities comprising:
a) t(4;14)(p16;q32);
b) t(14;16)(q32;q23);
c) dell7p;
d) t(4;14)(p16;q32) and t(14;16)(q32;q23);
e) t(4;14)(p16;q32) and dell7p;
f) t(14;16)(q32;q23) and dell7p;
g) t(4;14)(p16;q32), t(14;16)(q32;q23) and dell7p;
h) dell3; or
i) t(11;14)(q13;q32).

26) The method of claim 4, wherein the subject has refractory or relapsed multiple myeloma.

27) The method of claim 4, wherein the anti-CD38 antibody is a non-agonistic antibody.

28) A method of enhancing daratumumab-mediated antbody-dependent cellular phagocytosis (ADCP) in a subject, comprising administering to the subject daratumumab and cyclophosphamide, wherein cyclophosphamide enhances daratumumab-mediated ADCP.

29) The method of claim 28, wherein cyclophosphamide is administered at a dose of about 150-300 mg/m².

30) The method of claim 29, wherein daratumumab is administered at a dose of about 16 mg/kg.

31) The method of claim 30, wherein daratumumab and cyclophosphamide are administered the same day.

32) The method of claim 30, wherein cyclophospahmide is administered at a dose of about 150 mg/m².

33) The method of claim 30, wherein cyclophospahmide is administered at a dose of about 300 mg/m².

34) The method of claim 28, wherein the subject has a CD38-positive hematological malignancy.

35) The method of claim 34, wherien the CD38-positive hematological malignancy is multiple myeloma, acute lymphoblastic leukemia, diffuse large B-cell lymphoma, Burkitt's lymphoma, follicular lymphoma, mantle-cell lymphoma, acute myeloid leukemia, chronic lymphocytic leukemia, or combinations thereof.

36) The method of claim 35, wherein the CD38-positive hematological malignancy is multiple myeloma.
